(19) United States
(12) Patent Application Publication
(10) Pub. No.: US 2025/0367407 A1
(43) Pub. Date: Dec. 4, 2025

(54) Deflection Feedback Assemblies for Catheters and Sheaths

(71) Applicant: St. Jude Medical, Cardiology Division, Inc., Saint Paul, MN (US)

(72) Inventors: Sunil Gaddam, Carver, MN (US); Gregory Dakin, Edina, MN (US); Tim Marass, Minneapolis, MN (US); Troy Tegg, Elk River, MN (US)

(73) Assignee: St. Jude Medical, Cardiology Division, Inc., Saint Paul, MN (US)

(21) Appl. No.: 19/297,940

(22) Filed: Aug. 12, 2025

Related U.S. Application Data
(63) Continuation of application No. PCT/US2023/086267, filed on Dec. 28, 2023.
(60) Provisional application No. 63/445,489, filed on Feb. 14, 2023.
(60) Provisional application No. 63/599,721, filed on Nov. 16, 2023.

(51) Int. Cl. A61M 25/01; A61B 5/283; A61B 18/00; A61B 18/14

(52) U.S. Cl. CPC A61M 25/0136; A61M 25/0147; A61B 5/283; A61B 2018/00577; A61B 18/1492; A61M 2205/3327

(57) ABSTRACT

A deflectable catheter assembly including a handle, a catheter system, a method for determine deflection of a deflectable shaft section of the catheter. The catheter includes a handle coupled for deflecting the deflectable shaft section, and one or more pull wires to effect a deflection. The handle includes a housing, a deflection control coupled to the housing and operable to deflect the deflectable shaft section, and a deflection feedback assembly (e.g., a variable resistor) disposed within the housing. The deflection feedback assembly is coupled to the deflection control and configured to convert motion of the deflection control into a signal indicative of a degree of deflection of the deflectable shaft section. The deflection feedback assembly provides an active feedback regarding deflection of the deflectable shaft section to facilitate accurate positioning of the deflectable shaft section within a patient.

### CROSS REFERENCES TO RELATED APPLICATIONS

The present application is a Continuation of PCT Appln. No. PCT/US2023/086267 filed Dec. 28, 2023; which claims priority to U.S. Provisional Appln. Nos. 63/445,489 filed Feb. 14, 2023 and 63/599,721 filed Nov. 16, 2023; the full disclosures which are incorporated herein by reference in their entirety for all purposes.

### FIELD OF DISCLOSURE

This disclosure relates generally to an elongate catheter or sheath based cardiovascular medical device and related components. More particularly, this disclosure relates to a handle of a catheter or a sheath.

### BACKGROUND

Elongate catheter-based cardiovascular medical devices, such as electrophysiology (EP) catheters, can be used in a variety of diagnostic and/or therapeutic procedures to diagnose and/or correct medical conditions such as atrial arrhythmias, including for example, ectopic atrial tachycardia, atrial fibrillation, and atrial flutter. Arrhythmias can produce a variety of medical conditions including irregular heart rates, loss of synchronous atrioventricular contractions, and stasis of blood flow in a chamber of a heart, which can lead to a variety of other symptomatic and asymptomatic ailments and even death. The catheters may include multiple ring-shaped electrodes (or simply ring electrodes or electrodes) fixedly coupled to an elongate shaft section configured to achieve these diagnostic and/or therapeutic purposes. For example, some electrodes can be configured to transmit electrical signals from the heart anatomy for diagnostics (e.g., cardiac mapping), while other electrodes can be configured to impart resistive heating or irreversible electroporation (IRE) for therapeutics.

Radiofrequency (RF) ablation therapy can be conventionally used to treat various medical conditions. For example, RF ablation therapy may be used to treat cardiac arrhythmias. It is believed that the primary cause of atrial arrhythmia is stray electrical signals within the left or right atrium of the heart. An ablation catheter can be used to impart energy (e.g., radiofrequency energy, electroporation, cryoablation, lasers, high-intensity focused ultrasound, etc.) to create a lesion in the abnormal cardiac tissue, such that any undesirable electrical pathways within the heart can be potently limited or prevented.

Electroporation is a non-thermal ablation technique in which an electric field is applied to tissue to induce pore formation in cellular membranes. The electric field from electrode(s) can be applied in a pulse train of relatively short duration pulses that last, for example, from a nanosecond to several milliseconds. When electroporation is applied to tissue in an in vivo setting, the cells in the tissue are subjected to a trans-membrane potential to induce the pore formation in the cellular membranes. Electroporation may be reversible (i.e., the induced pores are temporarily formed) or irreversible (i.e., the induced pores remain open and induce cellular necrosis). In the field of cardiovascular diseases, irreversible electroporation can be used to induce cell necrosis in the cardiac tissues that may cause any undesirable electrical pathways within the heart, thereby achieving similar, and possibly superlative, therapeutics to conventional RF ablation.

An elongate medical device can be configured to provide an access to the heart anatomy and conduct relevant medical procedures (e.g., RF ablation, irreversible electroporation, cardiac mapping, etc.). For example, a cardiovascular catheter generally includes multiple shaft sections, including a proximal shaft section, a deflectable shaft section, and a distal functional shaft section disposed at, and interconnected to, the distal end of the deflectable shaft section.

The proximal shaft section of an elongate catheter is generally coupled with a handle and interconnected with the deflectable shaft section of the catheter. The deflectable shaft section of the catheter includes a pull ring or a deflection spine (e.g., planarity members for maintaining planarity of deflection) disposed at its distal end and one or more pull wires coupled to the pull ring, where the pull wire(s) passes through the proximal shaft section and then coupled to an actuation mechanism residing within the handle. Therefore, steering forces imposed at the handle can be effectively transmitted through the proximal shaft section to properly deflect or curve the deflectable shaft section in different orientations, such that the deflectable shaft section, including various related functional components (e.g., electrodes, sensors, etc.), can be desirably positioned within the heart anatomy for intended medical procedures. Examples of catheters with different shaft sections, in particular distal electrode shaft sections comprising ring electrodes, are disclosed in U.S. Pat. Nos. 5,524,337, 5,855,552, and 6,032,061, and 7,914,515 which are incorporated herein by reference in their entirety.

### BRIEF SUMMARY OF THE DISCLOSURE

The present disclosure relates to catheters used during medical procedures such as, for example, diagnostic and therapeutic procedures to detect and/or correct medical conditions such as atrial arrhythmias (e.g., ectopic atrial tachycardia, atrial fibrillation, and atrial flutter). A catheter includes an elongate shaft coupled to a handle configured to deflect a deflectable shaft section of the catheter. For example, the handle can include an actuator operable to effect deflection of the deflectable shaft section. When such deflectable shaft section is positioned within a human body, a degree of deflection achieved by moving the actuator is not visible. In order to facilitate accurate positioning of the deflectable shaft section or a tip of the catheter with respect to an area of interest (e.g., a point on a tissue within a heart), the handle herein includes a deflection feedback assembly. The deflection feedback assembly can be configured to output a signal indicative to a degree of deflection of the deflectable section. In some embodiments, a graphical user interface can be configured to display a degree of deflection (e.g., a shape, an amount, a direction, etc.) of the deflectable shaft section so that an operator can use a visual representation as a guide to accurately position the distal shaft section with respect to the area of interest.

Accordingly, in one aspect, a deflectable catheter assembly is described. The deflectable catheter assembly includes an elongate catheter shaft, a handle coupled to the elongate catheter shaft, and one or more pull wires. The elongate catheter shaft includes a deflectable shaft section. The handle includes a housing, a deflection control, and a deflection feedback assembly. The deflection control is coupled to the housing via one or more pull wires and operable to deflect the deflectable shaft section. The deflection feedback assembly is disposed within the housing. The deflection feedback assembly is coupled to the deflection control and configured to convert motion of the deflection control into a signal indicative of a degree of deflection of the deflectable shaft section. The one or more pull wires are coupled to the deflection control and extend through the elongate catheter shaft. The one or more pull wires are operable to induce deflection of the deflectable shaft section. In many embodiments, the deflection feedback assembly includes an electrical wire extending proximally away from the elongate catheter shaft.

In many embodiments, the deflection feedback assembly includes a variable resistor configured to vary resistance values upon operation of the deflection control. The variable resistor is coupled to the deflection control such that a first position of the deflection control corresponds to a first resistance value of the variable resistor and a second position of the deflection control corresponds to a second resistance value of the variable resistor. The first resistance value corresponds to a first degree of deflection of the deflectable shaft section; and the second resistance value corresponds to a second degree of deflection of the deflectable shaft section.

In some embodiments, the variable resistor includes a resistance element, a movable contact configured to move along the resistance element in response to operation of the deflection control to cause a change in a resistance value of the resistance element, a first terminal coupled to the resistance element to pass in an input electrical current through the resistance element, and a second terminal coupled to the movable contact. The second terminal is configured to output an electrical current indicative of the degree of deflection based on the resistance value corresponding to a position of the movable contact along the resistance element.

In some embodiments, the variable resistor is a rotary variable resistor. The resistance element is an arc-shape resistance element and the movable contact is radially movable along the resistance element in response to operation of the deflection control to cause a change in a resistance value of the resistance element. As an example, the deflection feedback assembly comprises a variable resistor having at least partial circular shape.

In some embodiments, the variable resistor is a linear variable resistor. The resistance element is a straight or a linear resistance element and the movable contact is slidable along the resistance element in response to operation of the deflection control to cause a change in a resistance value of the resistance element.

In many embodiments, the degree of deflection is characterized by a deflection amount and a deflection direction. The degree of deflection of the deflectable shaft section is determined based on the signal from the deflection feedback assembly.

In many embodiments, the handle further includes an inner platform disposed within the housing of the handle. The inner platform includes a first side and a second side opposite to the first side. The deflection control and the one or more pull wires are coupled to the first side of the inner platform, and the deflection feedback assembly is disposed on the second side of the inner platform. The deflection feedback assembly has a form factor corresponding to a shape of the second side of the inner platform and a thickness configured to fit within a space between the second side of the inner platform and an interior surface of the housing of the handle.

In some embodiments, the handle further includes a chassis slidable within the housing of the handle. The one or more pull wires are coupled to the chassis to cause deflection of the deflectable shaft section as the chassis is moved, and the deflection feedback assembly is coupled to the chassis such that the movement of the chassis is converted to the signal indicative of the degree of deflection.

In an example, the deflectable catheter assembly is bidirectional, where deflectable shaft section is configured to deflect in a first direction or an opposite second direction with respect to a home position by rotating the deflection control in one direction or an opposite direction. In another example, the deflectable catheter assembly is unidirectional, where the deflectable shaft section is configured to deflect in only one direction with respect to a home position by operating the deflection control.

In some embodiments, the deflection feedback assembly includes at least one of a magneto-resistor configured to cause change in a resistance upon rotating the deflection control. In some embodiments, the deflection feedback assembly includes a printed circuit board comprising a contact coupled to the deflection control. The printed circuit board configured to receive, via the contact, a position of the deflection control and convert the position to the degree of deflection of the deflectable shaft section.

In another aspect, a bidirectional deflectable catheter assembly is described. The bidirectional deflectable catheter assembly includes an elongate catheter shaft, a handle coupled to the elongate catheter shaft, and one or more pull wires. The elongate catheter shaft includes a deflectable shaft section. The handle can include a housing, a rotatable knob coupled exterior to the housing and operable to deflect the deflectable shaft section; and a variable resistor configured to vary resistance values upon rotation of the rotatable knob. Each resistance value corresponds to a degree of deflection of the deflectable shaft section. The one or more pull wires is coupled to the rotatable knob and extending through the elongate catheter shaft and operable to induce deflection of the deflectable shaft section in towards a first side or a second side with respect to a home position. The variable resistor further includes an electrical wire extending proximally away from the elongate catheter shaft.

In many embodiments, the handle further includes an inner platform disposed within the housing of the handle. The inner platform includes a first side and a second side opposite to the first side. The rotatable knob and the one or more pull wires are coupled to the first side of the inner platform, and the variable resistor is disposed on the second side of the inner platform.

In many embodiments, the variable resistor has a form factor corresponding to a shape of the second side of the inner platform and a thickness configured to fit within a space between the second side of the inner platform and an interior surface of the housing of the handle. The variable resistor is a rotary variable resistor including an arc-shape resistance element, a movable contact radially movable along the arc-shape resistance element in response to rotating of the rotatable knob to cause a change in a resistance value along the arc-shape resistance element, a first terminal coupled to the arc-shape resistance element to pass an input electrical current through the arc-shape resistance element, and a second terminal coupled to the movable contact, the second terminal configured to output an electrical current indicative of the degree of deflection based on the resistance value corresponding to a position of the movable contact along the resistance element. As an example, the variable resistor has at least partial circular shape. In one example, the variable resistor is disposed exterior to the housing and coupled to the rotatable knob by a serrated shaft.

In yet another aspect, a catheter system includes an elongate catheter shaft, a handle coupled to the elongate catheter shaft, one or more pull wires, a connector, and a controller. The elongate catheter shaft includes a deflectable shaft section. The handle includes a housing having a distal end and a proximal end, the distal end being configured to receive the elongate catheter shaft, a deflection control coupled to the housing and operable to deflect the deflectable shaft section, and a deflection feedback assembly disposed within the housing and coupled to the deflection control. The deflection feedback assembly is configured to provide output signals indicative of deflection amounts of the deflectable shaft section in response to operation of the deflection control. The one or more pull wires are coupled to the deflection control and extending through the elongate catheter shaft and operable to induce deflection of the deflectable shaft section. The connector coupled at the proximal end of the housing and configured to pass one or more cables. The controller is coupled via the one or more cables of the connector to the handle and a display. The controller is configured to determine, based on deflection mapping data and the output signals, the deflection amounts of the deflectable shaft section, the deflection mapping data being a predetermined relationship between the output signals and the deflection amounts.

In many embodiments, the output signals are characterized by a variable parameter. The deflection mapping data includes a first parameter value corresponding to a first deflection amount of the deflectable shaft section; and a second parameter value corresponding to a second deflection amount of the deflectable shaft section. The variable parameter is at least one of: an electrical resistance, an electrical current, a voltage, or a magnetic strength.

In some embodiments, the deflection feedback assembly is a variable resistor or a variable inductor. In an embodiment, the variable resistor is a rotary variable resistor. In some embodiments, the variable resistor includes a resistance element, a movable contact configured to move along the resistance element in response to operation of the deflection control to cause a change in a resistance value of the resistance element, a first terminal coupled to the resistance element to pass in an input electrical current through the resistance element, and a second terminal coupled to the movable contact. The second terminal is configured to output an output electrical current indicative of the degree of deflection based on the resistance value corresponding to a position of the movable contact along the resistance element.

In some embodiments, the controller is configured to send an input probing current to the first terminal of the variable resistor, receive, via the second terminal of the variable resistor, an output current after the input probing current passes through the resistance element, and determine the degree of deflection of the deflectable shaft section based on the output signal and the deflection mapping data.

In yet another aspect, a method for determining a shape of a deflectable shaft section of an elongate catheter shaft coupled to a handle is described. The method includes receiving, via the handle, a first degree of deflection of the deflectable shaft section of the elongate catheter shaft in a body of a patient. The handle includes a housing, a deflection control operable to deflect the deflectable shaft section, the deflection control in a first position, the first position corresponding to the first degree of deflection of the deflectable shaft section, and a deflection feedback assembly disposed within the housing and configured to convert a position of the deflection control into a signal indicative of a degree of deflection of the deflectable shaft section.

The method further includes receiving, via the deflection feedback assembly, a first signal indicating that the deflection control is in the first position. Further, the method includes receiving a second position of the deflection control. The second position corresponds to a second deflection of the deflectable shaft section. Further the method includes receiving, via the deflection feedback assembly, second signal indicating that the deflection control having the second deflection. Further, the method includes generating, via a processor, a visual representation of the deflectable shaft section. The visual representation indicating the deflectable shaft section having the second degree of deflection.

In some embodiments, the method further includes determining, using deflection mapping data, the first degree of deflection and the second degree of deflection based on the first signal associated with the first position and the second signal associated with the second position of the deflection control. The deflection mapping data indicates a relationship between the signals and the deflection amounts.

In some embodiments, the method further includes generating the deflection mapping data based on signals of the deflection feedback assembly, positions of the deflection control, and degrees of deflection of the deflectable shaft section. For example, generating the deflection mapping data includes: measuring, via the deflection feedback assembly, a signal at each of a number of pre-determined positions of the deflection control on the handle; measuring a deflection amount of the deflectable shaft section at each of the pre-determined positions; and generating a deflection mapping table relating each of the signals and each associated deflection amounts, and linking each signal to each associated predetermined position.

The handle with the deflection feedback assembly of the present disclosure provides several advantages. For example, a position of a deflection control (e.g., an angular position of a rotatable knob) can be converted into a signal for predicting a degree of deflection e.g., a shape of the deflectable shaft section. Predicting the degree of deflection can facilitate eliminating or limiting a need for fluoroscopy in electrophysiology procedures. For example, fluoroscopy provides a visualization of the deflectable shaft section and its disposition with respect to a point of interest within a patient. However, fluoroscopy can be undesirable and may require pausing the procedure to perform an extra step adding extra time to the procedure. Hence, instead of fluoroscopy, predicted degree of deflections can be rendered for an operator during an EP procedure. The deflection feedback assembly is an addition of electro-mechanical feature within a handle, rather than in a catheter shaft, that produces data from the catheter to approximate a degree of deflection.

Furthermore, as an example, any change in a knob rotation to deflect the deflectable shaft section can be captured by a variable resistor, which can be an example of the deflection feedback assembly. The variable resistor captures such knob rotation change as a measurable resistance value. The changes in the resistance values can be probed by the system and mapped back to an approximate a shape and direction of the deflection using calibration data. Such calibrated mapping data provides further advantages. For example, rotary variable resistors and the deflectable shaft can have part-to-part variation. To reduce the uncertainty in deflection feedback, each catheter can be calibrated to record resistance values at nominal, intermediate and extreme positions, a conversion coefficient like ohms per degree, and the calibration data can be saved in a memory component within the device. The measured resistance, voltage or another parameter can be interpreted per the calibration data, specific to a particular catheter, to improve predictions of degree of deflections.

The handle with the deflection feedback assembly can solve serval problems that may be encountered when using existing sensors for deflection shape predictions. For example, if additional magnetic sensors e.g., placed proximal to a pull-ring is used, these additional sensors can substantially increase cost and complexity of a catheter shaft assembly. On the other hand, the use of a variable resistor in the handle has minimal impact on the device cost and has no impact on the catheter shaft design. Additionally or alternatively, a catheter may lose its ability to deflect a distal end of the deflectable catheter shaft over time. Currently, the loss of deflection cannot be detected without physician's help. As another example, a catheter may partially lose its ability to deflect or the ability to deflect to the fullest extent. This loss in performance may go unnoticed affecting the quality of the procedure and possibly extending the procedure duration. On the other hand, the deflection feedback assembly can advantageously be used to indicate issues e.g., loss of deflection. For example, position data (e.g., from magnetic sensors) and the deflection feedback signal can be analyzed. If a change in deflection signal does not result in a reasonable change in position indicated by the magnetic sensors, an alert or a warning can be generated to inform the operator of potential loss in ability to deflect the distal end of the deflectable catheter.

In yet another aspect, a deflectable catheter assembly includes an elongate catheter shaft, one or more pull wires, and a control handle. The elongate catheter shaft includes a deflectable shaft section. Each of the one or more pull wires extends through the elongate catheter shaft and is actuatable to induce deflection of the deflectable shaft section. The control handle is coupled to the elongate catheter shaft. The control handle includes one or more slide members, a rotatable deflection control knob, and a housing. The one or more slide members are coupled with the one or more pull wires. At least one of the one or more slide members includes a visual position indicator configured to indicate one or more reference positions of the one or more slide members. The rotatable deflection control knob is drivingly coupled with the one or more slide members and rotatable to induce translation the one or more slide members to actuate the one or more pull wires. The one or more slide members are slidably disposed in the housing. The housing includes an indication port through which the visual position indicator is visible when the one or more slide members are in at least one of the one or more reference positions of the one or more slide members.

In some embodiments of the deflectable catheter assembly, the visual position indicator is visible through the indication port when the one or more slide members are in a neutral position in which the one or more pull wires do not induce deflection of the deflectable shaft section in the absence of any external contact forces being applied to the deflectable shaft section. In such embodiments, the visual position indicator can be configured to not be visible through the indication port when the one or more slide members are in a translated position in which the one or more pull wires induce deflection of the deflectable shaft section.

In some embodiments of the deflectable catheter assembly, the visual position indicator includes deflection magnitude indications. In some embodiments, each of the deflection magnitude indications is visible through the indication port when the one or more slide members are positioned to induce an amount of deflection of the deflectable shaft section corresponding to the deflection magnitude indication.

In some embodiments of the deflectable catheter assembly, the one or more slide members include two slide members. In some embodiments, the one or more pull wires include two pull wires, each of the two pull wires is coupled to a respective one of the two slide members, and one of the two slide members includes the visual position indicator.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale.

Any values dimensions illustrated in the accompanying graphs and figures are for illustration purposes only and can or cannot represent actual or preferred values or dimensions. Where applicable, some or all features cannot be illustrated to assist in the description of underlying features.

FIG. 1 illustrates a bidirectional deflectable catheter that can include a deflection feedback assembly, in accordance with many embodiments.

FIG. 2 shows an exploded view of the handle of the catheter or FIG. 1.

FIG. 3A illustrates an example of the deflection feedback assembly (in a first configuration) that can be employed in the catheter of FIG. 1.

FIG. 3B illustrates a first deflected shape of a deflectable shaft section indicated by the deflection feedback assembly in the first configuration of FIG. 3A.

FIG. 3C illustrates the deflection feedback assembly of FIG. 3A in a second configuration.

FIG. 3D illustrates a second deflected shape of a deflectable shaft section indicated by the deflection feedback assembly in the second configuration of FIG. 3C.

FIG. 3E illustrates another example of a deflection feedback assembly that can be employed in the catheter of FIG. 1.

FIG. 3F illustrates a resistance disk assembly of the deflection feedback assembly of FIG. 3E.

FIG. 3G illustrates a contact assembly of the deflection feedback assembly of FIG. 3E.

FIG. 3H illustrates a rotation knob assembly of the deflection feedback assembly of FIG. 3E.

FIG. 3I illustrates a 0 degree orientation configuration of the deflection feedback assembly of FIG. 3E.

FIG. 3J illustrates a 180 degree orientation configuration of the deflection feedback assembly of FIG. 3E.

FIG. 3K illustrates a 360 degree orientation configuration of the deflection feedback assembly of FIG. 3E.

FIG. 3L illustrates a 520 degree orientation configuration of the deflection feedback assembly of FIG. 3E.

FIG. 4 shows an exploded view of an example handle for a unidirectional deflectable catheter that can include a deflection feedback assembly, in accordance with embodiments.

FIG. 5 illustrates a variable linear resistor that can be employed in a deflection feedback assembly, in accordance with embodiments.

FIG. 6A shows a flow chart of a method of determining deflection shape of a deflectable catheter, in accordance with embodiments.

FIG. 6B shows a flow chart of a method of generating mapping data for use in determining deflection shape of a deflectable catheter, in accordance with embodiments.

FIG. 7 illustrates another control handle for a bidirectional deflectable catheter, in accordance with embodiments.

FIG. 8 shows an exploded view of the control handle of FIG. 7.

FIG. 9 shows an exploded view of right and left slides of the control handle of FIG. 7.

FIG. 10 shows a photograph of a partially disassembled configuration of the control handle of FIG. 7 illustrating an indication port and a visual position indicator attached to one of the slides.

FIG. 11 shows a photograph of the control handle of FIG. 10 illustrating visibility of the indicator through the indication port when the slides are in neutral positions.

FIG. 12 shows a photograph of a partially disassembled configuration of the control handle of FIG. 7 illustrating an indication port and an indicator attached to one of the slides with the slides displaced from the neutral positions.

FIG. 13 shows a photograph of the control handle of FIG. 13 illustrating non-visibility of the indicator through the indication port when the slides are displaced from the neutral positions.

FIG. 14 illustrates a visual position indicator for a deflection feedback assembly that includes deflection magnitude indications, in accordance with embodiments.

FIG. 15 illustrates a deflection feedback assembly that includes a neutral position indicator light, in accordance with embodiments.

FIG. 16 illustrates the use of a catheter that includes a deflectable section and a handle operable to articulate the deflectable section in a procedure on a patient, in accordance with embodiments.

FIG. 17 shows a schematic and block diagram view of a system for electroporation therapy, in accordance with embodiments.

FIG. 18 shows an example of a visual representation of a predicted deflected shape of a deflectable catheter and an indication of the amount of handle rotation displayed on a graphical user interface.

### DETAILED DESCRIPTION OF THE DISCLOSURE

The description set forth below in connection with the appended drawings is intended as a description of various embodiments of the disclosed subject matter and is not necessarily intended to represent the only embodiment(s). In certain instances, the description includes specific details for the purpose of providing an understanding of the disclosed embodiment(s). However, it will be apparent to those skilled in the art that the disclosed embodiment(s) can be practiced without those specific details. In some instances, well-known structures and components can be shown in block diagram form in order to avoid obscuring the concepts of the disclosed subject matter.

The present disclosure provides a catheter suitable for use in the human vasculature for known medical procedures, such as cardiac ablation, cardiac mapping, irreversible electroporation, etc. Such a catheter includes an elongate shaft with a deflectable shaft section. The deflectable shaft section is deflectable via a deflection control provided on a handle coupled to the elongate shaft. For example, the deflection control can be a rotatable knob or a slider. According to embodiments of the present disclosure, a deflection related feedback of the catheter can be achieved with a deflection feedback assembly (e.g., a deflection feedback assembly that includes a variable resistor) coupled to the deflection control of the handle. The deflection feedback assembly can convert a position of the deflection control (e.g., an angular position of a rotatable knob or a linear position of a slider), into a signal indicative of a degree of deflection of the deflectable shaft section of the catheter. For example, the signal can be an electrical resistance value corresponding to the position of the deflection control. Further, the signal can be transmitted to a calibrated mapping system. The calibrated mapping system is configured to convert the deflection related signal to the degree of deflection (e.g., an amount and direction of deflection) of the deflectable shaft section of the catheter. For example, the calibrated mapping system can read and translate the electrical resistance values into an amount of deflection, a deflection shape, or other deflection related parameters and render the shaft shape on a graphical user interface. The calibrated mapping system can be represented as a mapping table. As different catheters can have different material compositions or inherent variations, providing calibrated data associated with a particular catheter and its handle can reduce uncertainty and improve reliability of deflection related feedback. It is contemplated that the described features may be incorporated into any number of catheters or introducers as would be appreciated by one of ordinary skill in the art.

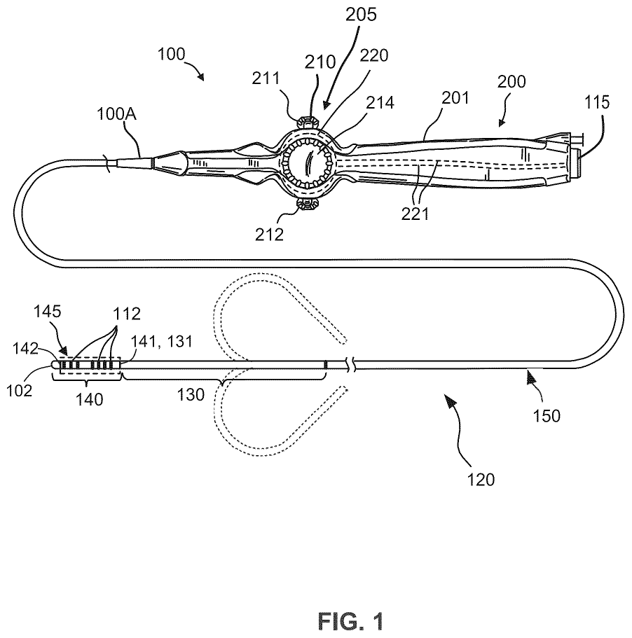

Referring now to the figures, in which like reference numerals refer to the same or similar features in the various views, FIG. 1 shows a catheter 100 with a handle 200, in accordance with many embodiments. The catheter 100 includes an elongate shaft 120 comprising a deflectable shaft section 130 and a distal shaft section 145 onto which electrodes 112 are mounted e.g., via mechanical swaging. In many embodiments, a proximal end 141 of the distal shaft section 145 can be adherently attached to a distal end of the deflectable shaft section 130 of the catheter 100. The distal shaft section 145 has a distal end 142 that may be adherently attached to other functional shaft sections (for example, for adopting an ablation tip & assembly and for accommodating sensors), such that the distal shaft section with electrodes, when integrated with the other functional shaft sections, may be alternatively identified as a distal functional shaft assembly (FSA) 140 of the catheter 100. A proximal portion 100A of the proximal shaft section 150 is coupled to the handle 200.

In many embodiments, the elongate shaft 120 has a composite, hollow shaft structure. The elongate shaft 120 includes various shaft sections with varying mechanical properties (e.g., stiffness, rigidity, flexibility, etc.), and/or may contain different electrical and functional components or assemblies, such as conductors or wires, magnetic sensors, optical sensors, etc. The elongate shaft 120 can be made of same or different materials to collectively achieve a desired mechanical performance for a particular shaft section of the catheter 100.

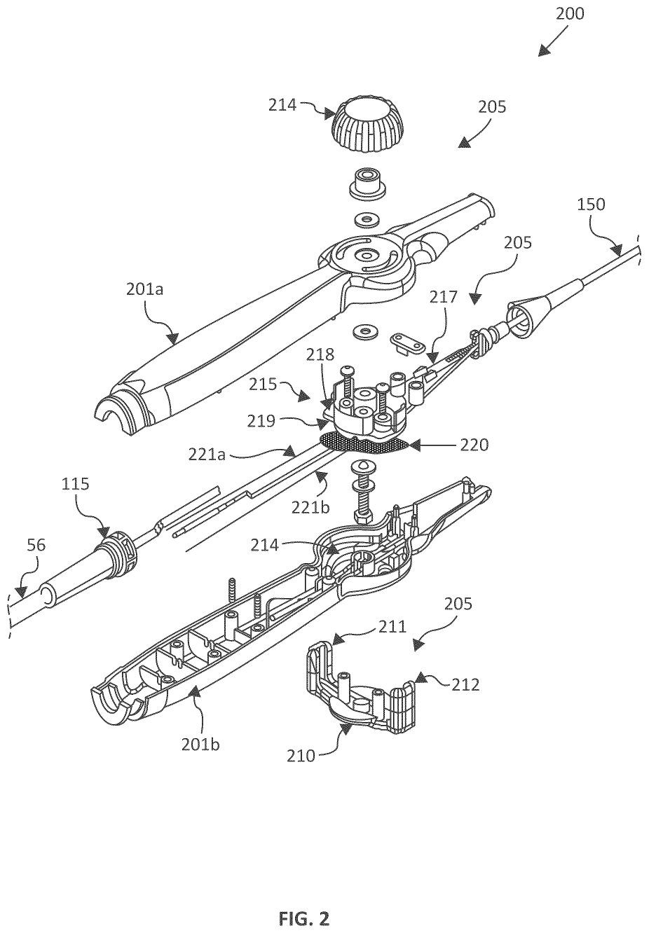

FIG. 1 illustrates the handle 200 coupled to the elongate catheter shaft 120 and FIG. 2 further illustrates an exploded view of the handle 200. In the illustrated embodiments, the handle 200 can include a deflection control 205 (e.g., a rotatable actuator or a slider) operable to selectively curve the deflectable shaft section 130 of the catheter 100 together with the distal shaft section 145 with electrodes 112 and other applicable functional shaft sections. For example, the deflectable shaft section 130, along with the distal shaft section 145, is configured to be selectively deflected in either of two directions as illustrated to accommodate navigation of the elongate shaft 120 through a patient's vasculature and/or positioning/orientation of the distal shaft section 145 of the catheter 100 within the heart anatomy during a medical procedure.

In the illustrated embodiments, the deflection control 205 of the handle 200 can include an outer actuator 210 and an outer knob 214. The outer actuator 210 can include a first boss 211 and a second boss 212 that a user (e.g., an electrophysiologist or other clinician) operates to effect deflection of the deflectable shaft section 130 of the catheter 100. The outer knob 214 can be configured to rotate and create a sufficient amount of preloading or internal friction in the handle 200 to temporarily lock the distal end 102 of the catheter 100 in a specified deflected configuration.

In many embodiments, the handle 200 can further include a deflection feedback assembly 220 disposed within a handle housing 201. The deflection feedback assembly 220 can be coupled to a deflection control 205 (e.g., the outer actuator 210) of the handle 200. The deflection feedback assembly 220 can convert a motion of the deflection control 205 into a signal indicative of a degree of deflection of a deflectable shaft section 130 of a catheter. For example, the deflection feedback assembly 220 can be coupled to the outer actuator 210 to convert a rotational position to a degree of deflection of the deflectable shaft section 130.

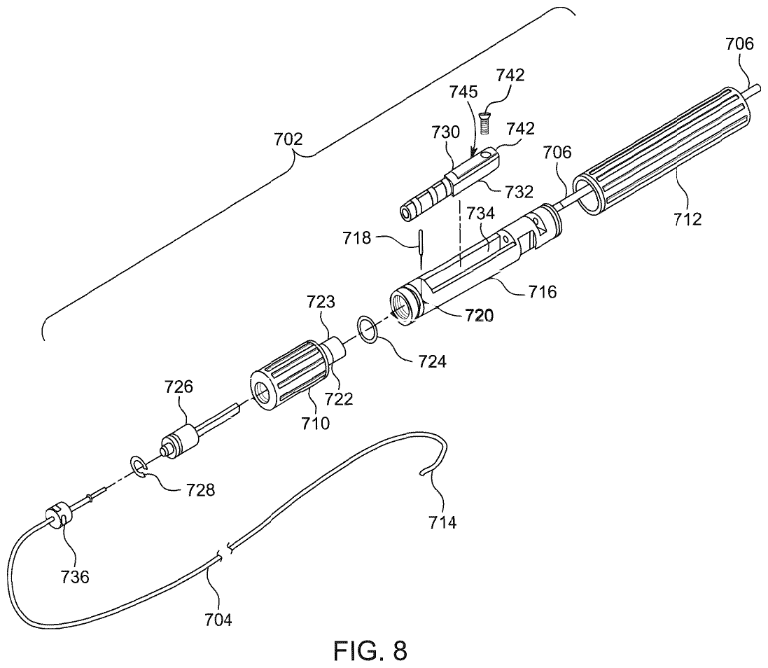

The deflection feedback assembly 220 can be coupled to wires 221 configured to receive an input signal and convey the deflection signal. The wires 221 can extend along a length of the handle 200 and extend towards the connector 115. The wires 221 can be accessed at the connector 115 and coupled to a controller or processor (e.g., 32 in FIG. 8) to analyze the deflection signal. In some embodiments, the wires 221 can be passed through an integrated cable (not illustrated). It can be understood that although the wires 221 are shown separated, the wires 221 may be routed through a common cable carrying other conducting wires used for communication and operations of the catheter via a controller or a processor (e.g., 32 in FIG. 8).

The deflection feedback assembly 220 can be configured to output a signal that varies in response to variable positions of the deflection control 205 (e.g., the outer actuator 210). For example, the signal can be characterized by a variable parameter such as a resistance, a current, a voltage, an impedance, a magnetic strength, or other variable mechanical, electrical, and/or magnetic parameter configured to vary in response to operation of the deflection control.

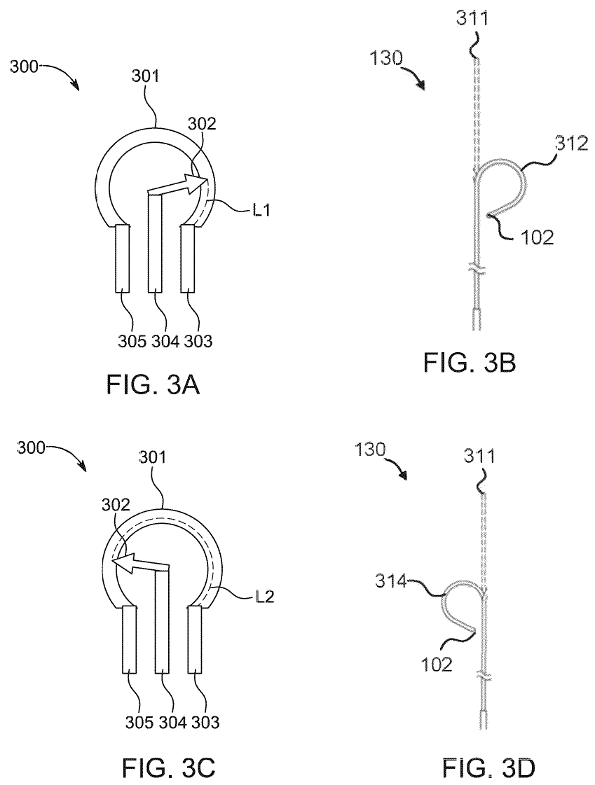

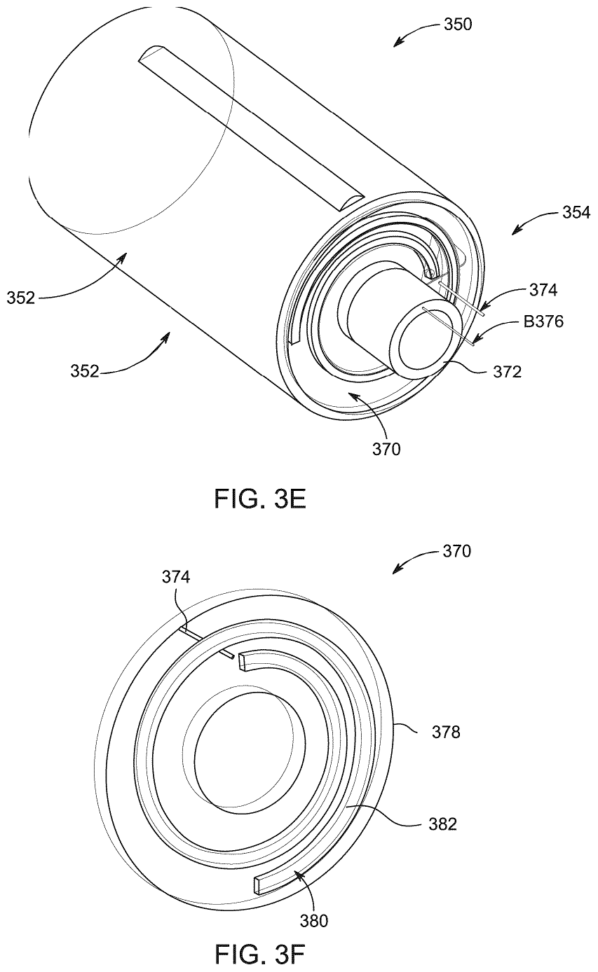

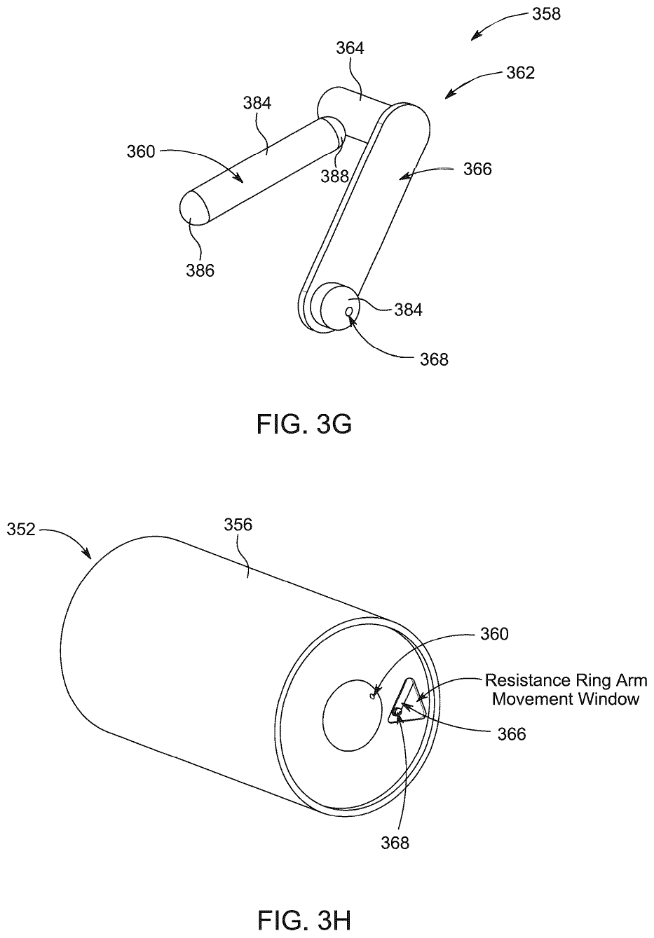

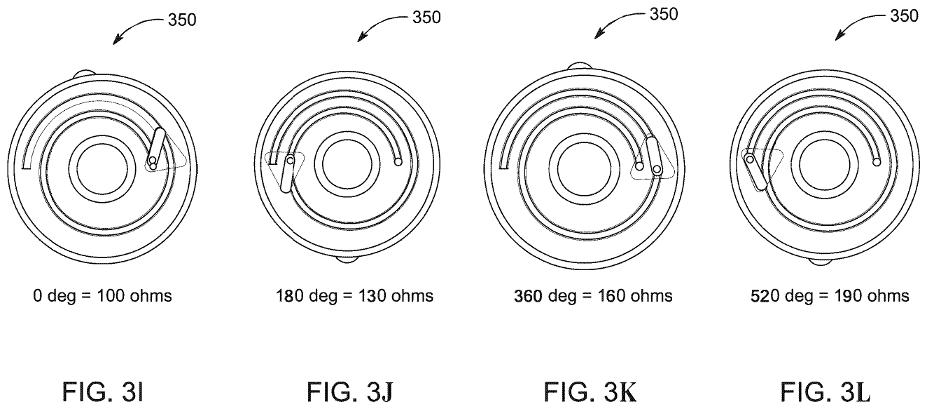

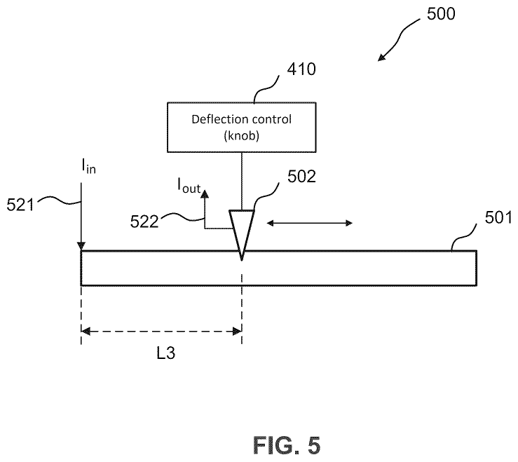

As an example, the deflection feedback assembly 220 can be a variable resistor configured to vary resistance values upon operation (e.g., rotation) of the deflection control 205 (e.g., the outer actuator 210). The variable resistor can be coupled to the deflection control 205 such that a first position of the deflection control 205 corresponds to a first resistance value of the variable resistor and a second position of the deflection control 205 corresponds to a second resistance value of the variable resistor. The deflection feedback assembly 220 can respond to rotary, sliding or other operation of the deflection control 205. Examples of the deflection feedback assembly 220 such as a rotary variable resistor are illustrated and further discussed with respect to FIG. 3A through FIG. 3L, without limiting the scope of the present disclosure and other implementation are possible. As another example, a deflection feedback assembly can be a linear variable resistor (see FIG. 5), a variable inductor, or other component.

An exploded view, in FIG. 2, of the handle 200 reveals several components including the handle housing 201, the deflection control 205 (e.g., 210 and 215), and the deflection feedback assembly 220. Further, FIG. 2 illustrates example positioning of the deflection feedback assembly 220 with respect to the deflection control 205 (e.g., 210 and 215) within the handle housing 201. As shown, the handle housing 201 can include an upper handle housing 201a and a lower handle housing 201b configured to accommodate several internal components. For example, the components can include an inner platform to which the deflection feedback assembly 220 can be coupled. In the illustrated embodiment, the inner platform can be an inner actuator 215 to which pull wires 217 can be coupled. The inner platform such as the inner actuator 215 can be pivotally sandwiched between the upper and lower handle housings 201a, 201b, and coupled to the outer actuator 210 on one side (e.g., bottom side in FIG. 2) of the inner platform (e.g., the inner actuator 215). Additionally or alternatively, the outer knob 214 can be coupled to an opposite other side (e.g., top side in FIG. 2) of the inner platform (e.g., the inner actuator 215). The outer actuator 210 and the inner actuator 215 cooperate to effectuate deflection of the deflectable shaft section 130 (in FIG. 2).

The inner actuator 215 can include a first surface 218 (e.g., a top surface) and a second surface 219 (e.g., a bottom surface) opposite the first surface. The first surface 218 can include projecting elements for coupling the outer knob 214, the pull wires 217, and other fastening means. The second surface 219 can be a substantially flat surface with holes to accommodate fasteners (e.g., screws). The inner actuator 215 and the components it supports are further discussed in detail in U.S. Pat. No. 9,861,788, which is incorporated by reference herein in its entirety.

Additionally, the handle housing 201 can be configured to accommodate wires or cables (e.g., conveying signals from electrodes 112) passing from the catheter 100 to the connector 115 of the handle 200. These internal components are packed in a compact manner so that the handle 200 can fit and be conveniently operable by one hand of a physician. Details of the handle 200 and its components are further discussed in detail in U.S. Pat. No. 9,861,788, which is incorporated by reference herein in its entirety.

To accommodate the internal components, the handle housing 201 can include one or more recesses, fastening related posts or holes, and other structural elements. For example, as shown, the lower handle housing 201b includes a recess 214 (e.g., approximately circular shaped) to accommodate the inner actuator 215 and related fasteners. Similarly, the upper handle housing 201a can include recesses (not shown) corresponding to shape of top side of the inner actuator 215.

In many embodiments, the deflection feedback assembly 220 can be shaped and sized to fit within a limited space available between internal components (e.g., 215) and the lower housing 201b. In other words, the deflection feedback assembly 220 can be configured to have a form factor conforming to a shape of an internal component (e.g., an inner platform such as the inner actuator 215). The form factor can correspond to the second side 219 of the inner actuator 215. For example, the form factor can be such that the deflection feedback assembly 220 does not interfere with other internal components such as screws, washers, cables, etc. Additionally, the deflection feedback assembly 220 can be thin enough (e.g., an electronic chip) to be disposed between the inner actuator 215 and the recess 214 within the lower handle housing 201b. In some embodiments, additional recess may be provided, sizes of the recesses may be enlarged, or the size of the internal components may be reduced to accommodate the deflection feedback assembly 220.

Although the present disclosure discusses the deflection feedback assembly as a resistance-type, it can be understood that the present disclosure is not limited to such resistance type deflection feedback assembly. For example, the deflection feedback assembly may include at least one of resistive or non-resistive components. For example, the deflection feedback assembly may include a magneto-resistor configured to cause change in a resistance upon rotating the deflection control 205. As another example, the non-resistive component may include inductors, capacitors, etc. In some embodiments, the deflection feedback assembly includes a printed circuit board comprising a contact coupled to the deflection control 205. The printed circuit board configured to receive, via the contact, a position of the deflection control and convert the position to the degree of deflection of the deflectable shaft section.

FIG. 3A illustrates a variable rotary resistor 300, which can be an example of the deflection feedback assembly 220. The variable rotary resistor 300 can be configured to be positioned under the inner actuator 215 (e.g., coupled to the bottom surface 219 in FIG. 2). In the illustrated embodiment, the variable rotary resistor 300 can include a resistance element 301, a movable contact 302, a first terminal 303, a second terminal 304, and/or a third terminal 305. Although, the example resistor is explained as resistor, it can be understood that the resistor 300 can be configured as a voltage divider having three contacts. The resistance element 301 can extend between the first terminal 303 and the third terminal 305. As an example, the resistance element 301 can be an arc-shape resistance element. The arch-shape can span between 0 to 180 degrees, 0 to 270 degrees, or other angular spans. The arc-shape can correspond to the recess 214 in the lower housing 201b and/or the second surface 219 of the inner actuator 215 (in FIG. 2). The resistance element 301 has a specified resistance per unit length and is electrically conductive. Accordingly, as a portion (e.g., an arcuate length) of the resistance element 301 between the first terminal 301 and the movable contact 302 increases, the resistance value increases.

The movable contact 302 can be configured to move along the resistance element 301 in response to operation of the deflection control 205 (e.g., the outer actuator 210 in FIG. 2) to cause a change in a resistance value of the resistance element 301. For example, the outer actuator 210 (in FIG. 2) can be coupled to an end (e.g., a tip portion (represented by an arrow) in contact with the resistance element 301) of the movable contact 302 so that the movable contact 302 moves directly or proportionally in response to rotation of the outer actuator 210. When the movable contact 302 is at a first position, a first portion (e.g., a first arcuate length L1) of the resistance element 301 has a first resistance value R1. When the movable contact 302 is at a second position, a second portion (e.g., a second arcuate length L2 in FIG. 3C) has a second resistance value R2. If the first arcuate length L1 is less than the second arcuate length L2, the resistance value R1 is less than resistance value R2.

The first terminal 303 can be coupled to the resistance element 301 to receive an input electrical current, which further passes through a portion of the resistance element 301. The second terminal 304 can be coupled to the movable contact 302 and configured to output a signal (e.g., an output electrical current different from the input electrical current). The output signal can be indicative of a degree of deflection based on the resistance value corresponding to a position of the movable contact 302 along the resistance element 301.

In operation, as the movable contact 302 moves away from the first terminal 303, the arcuate length of the resistance element 301 that carries the input current also increases. This in turn results in change in the output signal (e.g., an output electric current). Accordingly, when the outer actuator 210 (in FIG. 2) is rotated (e.g., by a physician or a clinician) to effectuate a desired deflection of the deflectable shaft section 130, the movable contact 302 moves along the resistor element 301 to generate an output signal that is indicative of a degree of deflection of the deflectable shaft section 130. Hence, when the deflectable shaft section 130 is within a human body and not visible to an operator (e.g., the physician or the clinician), the output signal can be used to predict a degree of deflection.

FIG. 3B illustrates a first degree of deflection 312 of the deflectable shaft section 130 of the catheter. The first degree of deflection 312 can correspond to the first position of the movable contact 302 shown in FIG. 3A. As discussed above, when the movable contact 302 is at the first position (in FIG. 3A), a first output signal can be generated. This output signal can indicate the first degree of deflection 312. For example, the output signal at the second terminal 304 can be used to determine that the first deflection 312 of the deflectable shaft section 130 is towards right and an amount of deflection is approximately 180 degrees or more measured with respect to a home position 311 of the deflectable shaft section 130, as shown.

FIG. 3C illustrate the variable rotary resistor 300 with the movable contact 302 in a second position. The second position can be achieved by moving the outer actuator 210 (e.g., in FIG. 2) to deflect the deflectable shaft section 130 in another direction (e.g., to left side). At the second position of the movable contact 302, a second output signal can be transmitted through the second terminal 304. The second output signal can correspond to a second output current received after passing through a second length L2 of the resistance element 301. The second output signal can indicate a second degree of deflection.

For example, FIG. 3D illustrates a second degree of deflection 314 of a deflectable shaft section 130 indicated by the variable rotary resistor 300 of FIG. 3C. The output signal caused by the second length L2 of the resistance element 301 can correspond to the second degree of deflection 134. For example, the output signal can indicate the deflectable shaft section 130 (in FIG. 3D) is deflected in a second direction (e.g., to the left side) by an amount −180 degrees with respect to the home position 311.

In the present disclosure, a degree of deflection can be characterized by one or more deflection related parameters such as an amount of deflection, a distance, a curvature, a direction, or other deflection related parameter without limiting the scope of the present disclosure. For example, an amount of deflection of the distal tip 102 of the deflectable shaft section 130 with respect to the home position 311 (in FIG. 3B and FIG. 3D) can be measured. As another example, a distance between the distal tip 102 with respect to the home position 311 can be measured. As another example, a curvature of the deflectable shaft section 130 can be measured. As another example, a direction (e.g., left or right side) of the with respect to the home position 311 can be measured.

FIG. 3E illustrates a deflection feedback assembly 350 that can be employed in a deflectable catheter, in accordance with embodiments. The deflection feedback assembly 350 includes a rotation knob assembly 352 (separately shown in FIG. 3H) and a variable resistance assembly 354 (separately shown in FIG. 3F). The rotation knob assembly 352 can be included in a handle assembly of a deflectable catheter that includes a deflectable shaft section. The rotation knob assembly 352 can be mounted to a handle housing of the handle assembly for rotation relative to the handle housing for actuating one or more pull wires (e.g., one or more pull wires 417) to change a deflected shape of the deflectable shaft section. The rotation knob assembly 352 includes a rotation knob 356 and a resistance contact assembly 358 mounted to the rotation knob 356. The variable resistance assembly 354 can be fixedly mounted to the handle housing so that rotation of the rotation knob assembly 352 relative to the handle housing rotates the resistance contact assembly 358 relative to the variable resistance assembly 354. The resistance contact assembly 358 includes a center spring contact 360 and a resistance arm assembly 362. The resistance arm assembly 362 includes a resistance arm shaft 364, a resistance ring arm 366, and a resistance ring contact 368. The resistance arm shaft 364 is rotationally mounted to the rotation knob 356 for rotation around a centerline of the resistance arm shaft 364. A first end portion of the resistance ring arm 366 is attached to the resistance arm shaft 364. A second end portion of the resistance ring arm 366 is offset laterally from the centerline of the resistance arm shaft 364. The resistance ring contact 368 is attached to a protrude from the second end portion of the resistance ring arm 366.

The variable resistance assembly 354 includes a resistance ring 370 (shown separately in FIG. 3F), a central contact shaft 372, a first signal wire 374, and a second signal wire 376. The resistance ring 370 includes a non-conductive disk-shaped base member 378 with a spiral-shaped recess 380 and a spiral-shaped variable resistor member 382 disposed within and extending along the length of the spiral-shaped recess 380. In the illustrated embodiment, the spiral-shaped variable resistor member 382 extends around a rotational axis of the rotation knob assembly 352 through 520 degrees. The spiral-shaped variable resistor member 382, however, can extend around the rotational axis of the rotation knob assembly 352 through any suitable number of degrees. A first signal wire 374 is connected to a first end of the spiral-shaped variable resistor member 382. The second signal wire is connected to the central contact shaft 372, which is made from an electrically conductive material.

The variable resistance assembly 354 is configured to produce a variable resistance between the first signal wire 374 and the second signal wire 376 indicative of the rotational orientation of the rotational knob assembly 352 relative to the handle housing. In the illustrated embodiment, the rotation knob assembly 352 is rotatable relative to the resistance ring 370 (which is mounted to the handle housing to prevent rotation of the resistance ring 370 relative to the handle housing) through 520 degrees. The resistance ring contact 368 is held in contact with the spiral-shaped variable resistor member 382 throughout the 520 degrees of rotation of the rotation knob assembly 352 relative to the handle housing. In the illustrated embodiment, the resistance ring contact 368 has an axial-symmetric guide surface 384 shaped to protrude into and interface with the spiral shaped recess 380 to control the radial position of the resistance ring contact 368 to keep the resistance ring contact 368 aligned with the spiral-shaped variable resistor member 382 throughout the 520 degrees. The resistance arm assembly 362 accommodates the variation in the radial position of the resistance ring contact 368 over the 520 degrees via relative rotation of the resistance arm assembly 362 relative to the rotation knob 356 around the centerline of the resistance arm shaft 364. Any suitable approach can be used to control the amount of force by which the resistance ring contact 368 is pressed against the spiral-shaped variable resistor member 382 over the 520 degrees of rotation. For example, a suitably configured compression spring can be interposed between the rotation knob 356 and the resistance arm shaft 364 to apply a suitable force to the resistance arm shaft 364 to bias the resistance arm assembly 362 towards the resistance ring 370. As another example, a suitably configured compression spring can be interposed between the resistance ring 370 and the rotation knob assembly 352 to apply a suitable force to the resistance ring 370 to bias the resistance ring 370 towards the resistance ring contact 368. The center spring contact 360 extends radially from the central shaft contact 372 and the resistance arm shaft 364. The resistance ring contact 368 is electrically connected to the second signal wire 376 via the resistance arm assembly 362, the central spring contact 360, and the central contact shaft 372, each of which are electrically conductive. The center spring contact 360 includes a conductive compression spring 384 and conductive end caps 386, 388. The conductive compression spring 384 is preloaded in compression and serves to keep the conductive end caps 386, 388 in contact with the central contact shaft 372 and the resistance arm shaft 364, respectively.

FIG. 3I, FIG. 3J, FIG. 3K, and FIG. L illustrate example configurations of the deflection feedback assembly 350. FIG. 3I illustrates a 0 degree orientation configuration of the deflection feedback assembly 350 in which the resistance arm contact 368 is contacted with the spiral-shaped variable resistor member 382 immediately adjacent to the end of the spiral-shaped variable resistor member 382 to which the first signal wire 374. To avoid inducing an excessive current draw through the spiral-shaped resistor member 382 in the 0 degree orientation configuration, a separate 100 ohm resistance is employed so that a resulting 100 ohm resistance is measured in the 0 degree orientation configuration.

FIG. 3J illustrates a 180 degree orientation configuration of the deflection feedback assembly 350 in which the resistance arm contact 368 is contacted with the spiral-shaped variable resistor member 382 180 degrees away from the end of the spiral-shaped variable resistor member 382 to which the first signal wire 374. The intervening 180 degree arc length of the spiral-shaped resistor member 382 adds 30 ohms to the separate 100 ohm resistance, thereby producing a resulting 130 ohm resistance in the 180 degree orientation configuration. FIG. 3K illustrates a 360 degree orientation configuration of the deflection feedback assembly 350 in which the resistance arm contact 368 is contacted with the spiral-shaped variable resistor member 382 360 degrees away from the end of the spiral-shaped variable resistor member 382 to which the first signal wire 374. The intervening 360 degree arc length of the spiral-shaped resistor member 382 adds 60 ohms to the separate 100 ohm resistance, thereby producing a resulting 160 ohm resistance in the 360 degree orientation configuration.

FIG. 3K illustrates a 520 degree orientation configuration of the deflection feedback assembly 350 in which the resistance arm contact 368 is contacted with the spiral-shaped variable resistor member 382 520 degrees away from the end of the spiral-shaped variable resistor member 382 to which the first signal wire 374. The intervening 520 degree arc length of the spiral-shaped resistor member 382 adds 90 ohms to the separate 100 ohm resistance, thereby producing a resulting 190 ohm resistance in the 520 degree orientation configuration. The 520 degree total rotation combined can be employed in any suitable manner. For example, for a deflectable catheter with a bi-directional deflectable shaft section, the rotation knob 356 can be rotated from a central neutral orientation 260 degrees in a first rotation direction to deflect the deflectable shaft section in a first deflection direction, and from the central neutral orientation 260 degrees in a second rotation direction (opposite to the first rotation direction) to deflect the deflectable shaft section in a second deflection direction (opposite to the first deflection direction).

In some embodiments, a catheter can be a bidirectional catheter in which a deflectable shaft section can be deflected in a first direction or a second direction with respect to a home position. In some embodiments, a catheter can be a unidirectional catheter, in which a deflectable shaft section can be deflection in only one direction. In an embodiment, the unidirectional catheter may include a handle having a slidable actuator. For example, a movable contact of a variable resistor can be slidable along a resistance element in response to operation of a deflection control to cause a change in a resistance value of the resistance element.

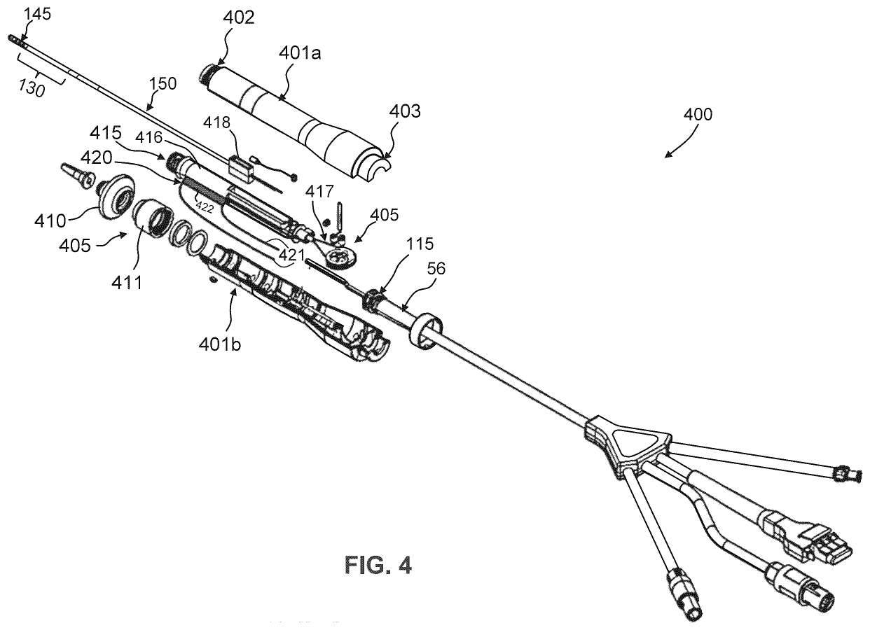

FIG. 4 is an exploded view of a unidirectional control handle 400 that includes another variation of a deflection feedback assembly for a deflectable catheter. The handle 400 can include an upper handle housing 401a and a lower handle housing 401b (collectively referred as a handle housing 401) and an outer actuator 410 coupled to a slidable actuator 415. The handle 400 can include a distal portion 402 and a proximal portion 403. The outer actuator 410 and the slidable actuator 415 can be interoperable to serve as a deflection control 405 of the deflectable shaft section 130. The outer actuator 410 can be coupled to an outer surface of the handle housing 401. In the illustrated embodiment, the outer actuator 410 can be a push-type knob coupled to the distal portion 402 of the handle 400. For example, the outer actuator 410 can be actuated by pushing, e.g., via thumb of an operator, to effect a deflection in the deflectable shaft section 130. In some embodiments, the handle 400 can further include an actuator lock 411 configured to lock the slidable actuator 415 and hence the deflection of the deflectable shaft section 130.

The slidable actuator 415 can be disposed internally between the upper handle housing 401a and the lower handle housing 401b. The slidable actuator 415 can include a chassis 416, one or more pull wires 417, and pull wire slides 418. The chassis 416 can be an elongate hollow shaft having a slide compartment. In an embodiment, the chassis 416 can be slidable within the housing 401 of the handle 400. The one or more pull wires 417 can be coupled to the chassis 416 to cause deflection of the deflectable shaft section 130 as the chassis is moved. In some embodiments, the slides 418 can be slidably disposed within the hollow portion of the chassis 416 and coupled to the pull wires 417. As the slides 418 move, in response to operation of the outer actuator 410, the pull wires 417 can cause deflection of the deflectable shaft portion 130.

In some embodiments, a deflection feedback assembly can be coupled to the chassis 416 such that the movement of the chassis 416 can be converted to a signal indicative of the degree of deflection of the deflectable shaft section 130. In the illustrated embodiment, the deflection feedback assembly can be a linear variable resistor 420 coupled to the handle housing 401 and the slidable actuator 415 (e.g., to the chassis 416). The linear variable resistor 420 can be configured such that as the chassis 416 slides the linear variable resistor 420 can output a signal indicative of a deflection of the deflectable shaft section 130. As an example, the linear variable resistor 420 can include a linear resistance element 422 coupled to the handle housing 401 and a movable contact may be coupled to the chassis 416 so that when the chassis 416 moves relative to the handle housing 401, the contact moves along the linear resistance element 422 causing a change in resistance. The change in resistance can be used to generate an output signal indicative of a deflection of the deflectable shaft section 130. Wires 421 can be configured to receive an input through one of the wires 421 and deliver an output through another one of the wires 421. The wires 421 can be electrical wires extending proximally away from the elongate catheter shaft 150.

FIG. 5 illustrates a variable linear resistor 500 as an example of the deflection feedback assembly of the handle 400 (in FIG. 4). The variable linear resistor 500 can include a linear resistance element 501, a movable contact 502, a first terminal 521, and/or a second terminal 522. In some embodiments, the linear resistance element 501 may have a linear taper (e.g., straight element). Alternatively, the linear resistance element 501 may have a non-linear taper. The movable contact 502 is slidable along the resistance element 501 in response to operation of the deflection control 405 (e.g., including the outer knob 410) to cause a change in a resistance value of the resistance element 501. The resistance element 501 has a specified resistance per unit area. The first terminal 521 can be coupled to one end of the linear resistance element 501 and configured to receive an input current (Iin) and pass it through the resistance element 501. The second terminal 522 can be coupled to the movable contact 502 and configured to output signal e.g., a function of resistance associated with the position of the movable contact 502 on the resistance element 501 and an output current (Iout). For example, the deflection control 405 (e.g., the outer knob 410) can be moved to cause a desired deflection of the deflectable shaft section 130 (e.g., in FIG. 4). In response, the movable contact 502 moves to a length L3 along the resistance element 501. When the input current is passed through this portion (of the length L3) of the resistance element 501, a first output signal is generated. The first output signal can be indicative of the deflectable shaft section 130. The output signal (e.g., a function of resistance and the output current) can be evaluated in a similar manner as discussed with respect to FIG. 3 to determine a degree of deflection (e.g., a deflection amount, a direction, etc.) of the deflectable shaft section.

The present disclosure is not limited to illustrated examples of the deflection feedback assembly and other variations are possible. In some examples, multiplexers/switches may be used to consolidate multiple connections including connections with the wires of the deflection feedback assembly and other wires passing through the catheter. Installing such multiplexers may be acceptable because high sampling rates may not be required for channels like temperature sensors or other sensors of the catheter. Thus, a same signal chain can be shared by multiple sensors. In this variation, a mapping system may be further developed that is capable of interpreting mixed signals. In some embodiments, the deflection feedback assembly can be an electro-magnetic, magnetic, impedance, electro-mechanical feedback or other elements that can be used to describe a position, an orientation, or a shape of the deflectable shaft section of the catheter with varying uncertainties. In some embodiments, the signals from the deflectable feedback component can be used to build algorithms, such as Bayesian models and Genetic algorithms, to combine all these sources to produce a shaft state prediction in an efficient way. As another application, a deflection feedback assembly can be incorporated into an introducer or a sheath to provide quantitative feedback regarding a degree of deflection at a distal end of the introducer or the sheath.

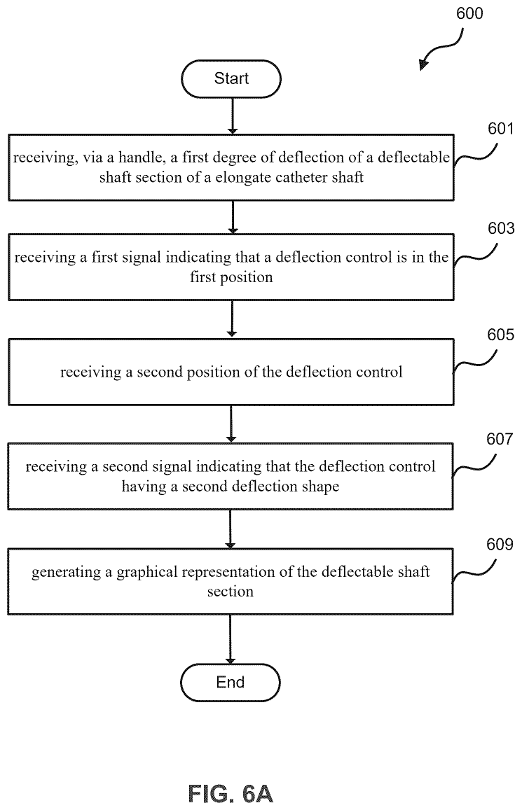

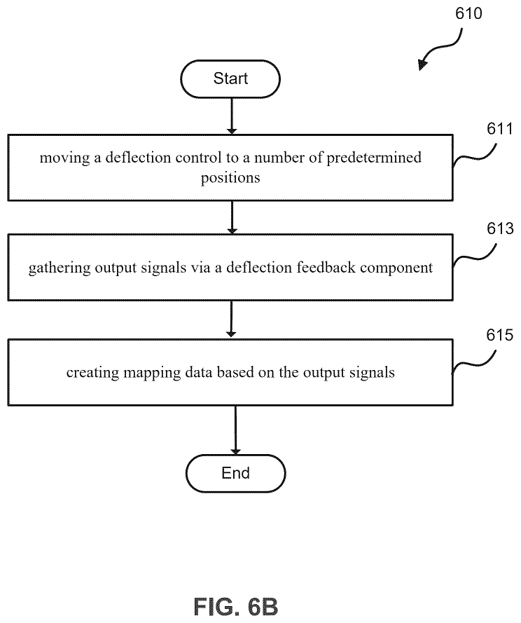

FIG. 6A is an example flow chart of a method 600 for determining deflection shape of a deflectable catheter, in accordance with examples of the present disclosure. As discussed herein, an elongate catheter shaft (e.g., 150) is coupled to a handle (e.g., a bidirectional or unidirectional handle) configured to effect deflection of the deflectable shaft section of the elongate catheter shaft. The handle can include a deflection feedback assembly configured to provide a deflection related signal which can be used to predict (e.g., via calibrated mapping data) a degree of deflection of the deflectable shaft section. The predicted degree of deflection (e.g., a shape or amount of deflection) can provide useful information during a medical procedure. For example, an extra fluoroscopy step which provides an actual visualization of the deflection of the deflectable shaft section within a human body can be eliminated. Instead, the predicted deflection shape can be used as a guide during the medical procedure. Thus, the deflection feedback assembly can advantageously save time, improve accuracy of positioning devices within the patient, and/or limit any harmful effects associated with fluoroscopy, among other advantages. The method 600 can include steps 601-609, further discussed in detail below.

Step 601 can involve receiving, via a handle, a first degree of deflection of the deflectable shaft section (e.g., 130) of the elongate catheter shaft in a body of a patient. As discussed herein the handle (e.g., 200, or 400) can include a housing, a deflection control (e.g., 205 or 405), and a deflection feedback assembly (e.g., 220 or 420). The deflection control is operable to deflect the deflectable shaft section (e.g., 130). For example, the deflection control can be moved to a first position by an operator (e.g., a physician or a clinician) to cause the first degree of deflection of the deflectable shaft section within the human body during a medical procedure. Accordingly, the first position corresponds to the first degree of deflection of the deflectable shaft section (e.g., 130). The deflection feedback assembly can be disposed within the housing and configured to convert a position of the deflection control into a signal indicative of a degree of deflection of the deflectable shaft section (e.g., 130). For example, the handle 200 can include the outer actuator 210 (an example of the deflection control) and the deflection feedback assembly 220. As the outer actuator 210 is moved to a first position (e.g., see FIG. 3(a)), a first degree of deflection of the deflectable shaft section 130 (e.g., as shown in FIG. 3(b)) can be achieved.

Step 603 can involve receiving, via the deflection feedback assembly, a first signal indicating that the deflection control is in the first position. For example, as shown in FIG. 2, the deflection feedback assembly 220 can include the resistance element configured to output a deflection signal indicating that the outer actuator 210 is at the first position. As further illustrated in example in FIG. 3, the variable rotary resistor 300 (which is an example of the deflection feedback assembly 220) outputs a signal at the second terminal 304 corresponding to the position of the movable contact 302, which is achieved by moving the outer actuator 210. In some embodiments, the first signal can be received by a processor (e.g., 32 in FIG. 8). The second signal can correspond to a current passed through the arcuate length L1, in FIG. 3(a).

Step 605 can involve receiving a second position of the deflection control. The second position can correspond to a second deflection of the deflectable shaft section (e.g., 130). For example, the outer actuator 210 (an example of the deflection control) can be moved to form the first position (e.g., see FIG. 3(a)) to the second position (e.g., see FIG. 3(c)). For example, an operator (e.g., a physician or a clinician) can rotate the outer actuator to deflect the deflectable shaft section 130 within the human body during a medical procedure. In some examples, the first degree of deflection and the second degree of deflection can be: both on one side of a home position, on either sides of the home position, or any other positions between extreme deflections.

Step 607 can involve receiving, via the deflection feedback assembly, a second signal indicating that the deflection control having the second deflection. For example, as shown in FIG. 2, the deflection feedback assembly 220 can include the resistance element configured to output a deflection signal indicating that the outer actuator 210 is at the second position. As further illustrated in example in FIG. 3(c), the variable rotary resistor 300 (which is an example of the deflection feedback assembly 220) outputs a signal at the second terminal 304 corresponding to the position of the movable contact 302, which is achieved by moving the outer actuator 210. In some embodiments, the second signal can be received by a processor (e.g., 32 in FIG. 8). The second signal can correspond to a current passed through the arcuate length L2, in FIG. 3(c).

In some embodiments, the method 600 can involve determining, using deflection mapping data, the first degree of deflection and the second degree of deflection based on the first signal associated with the first position and the second signal associated with the second position of the deflection control, the deflection mapping data indicating a relationship between the signals and the deflection amounts. As an example, the deflection mapping data can be generated based on signals of the deflection feedback assembly, positions of the deflection control, and degrees of deflections of the deflectable shaft section (e.g., 130).

As an example, FIG. 6B illustrates an example flow chart 610 for generating the deflection mapping data. It can be understood that the deflection mapping data can be generated before the step 601 so that the deflection mapping data can be stored (e.g., in a memory of a computer) and used during a medical procedure. In one embodiment, step 611 can involve moving the deflection control to a number of predetermined positions. Step 613 can involve gathering output signals via the deflection feedback assembly. For example, an output signal at each of a number of predetermined positions of the deflection control on the handle can be measured using the deflection feedback assembly. Further, a degree of deflection (e.g., an amount, shape, orientation, distance, etc.) of the deflectable shaft section (e.g., 130) at each of the predetermined positions can be measured. Step 615 can involve creating mapping data e.g., represented in form of a mapping table based on the gathered output signals. For example, each of the output signals from the deflection feedback assembly and each associated deflection amounts can be mapped or related to each other. Similarly, each signal can be linked to each associated predetermined position of the deflection control of the handle. Accordingly, when the deflection control is moved through different positions, a corresponding signal generated by the deflection feedback assembly can indicate a position of the deflection control. Further, the corresponding signal can be compared with the signals of the deflection mapping data to determine a degree of deflection (e.g., an amount, shape, orientation, distance, etc.). As discussed with respect to FIGS. 3 and 5, such data can be measured with respect to a home position of the deflectable shaft section.

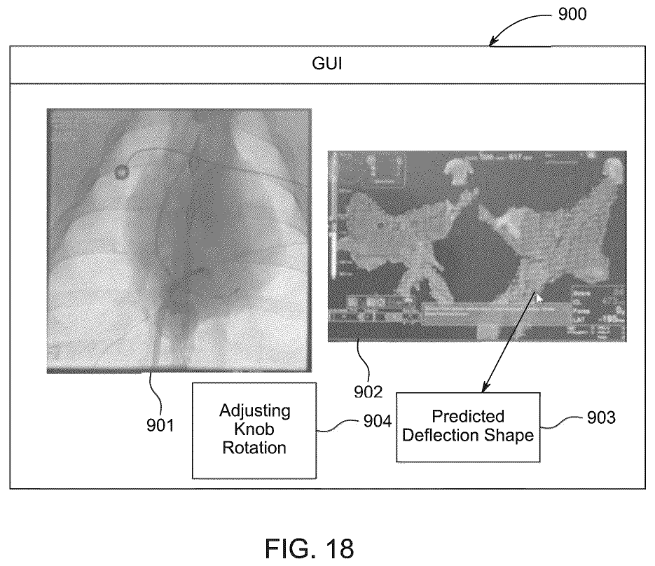

Referring back to FIG. 6A, step 609 can involve generating, via a processor, a visual representation of the deflectable shaft section (e.g., 130). The visual representation can indicate the deflectable shaft section (e.g., 130) having the first degree of deflection and/or the second degree of deflection. For example, a computer system or a processor (e.g., 32 in FIG. 8) can generate a visual representation on the display (e.g., 34b in FIG. 8). An example of the visual representation displayed on a graphical user interface 900 is shown in FIG. 18.

Referring to FIG. 18, the graphical user interface 900 can include one or more portions showing operating internals of a patient, or measurements of while operating internals of the patient. A first portion 901 can display a captured image (e.g., a fluoroscopy image) or other real-time image showing actual patient's internals and a deflectable shaft section within the patient. A second portion 902 can include a map indicating measurements (e.g., proximity measurements, electrical signals induced or flow through the tissue) at different points of a tissue or a part of the patient. A third portion 903 can include a predicted shape of the deflectable shaft section 130 that is positioned within the internals of the patient shown in the first portion 901 of the GUI 900. The predicted shape can be rendered on the third portion 903 of the GUI 900 in a 3D or other representations. Additionally or alternatively, a surrounding tissue may be rendered to provide a visual guide to a physician performing a medical procedure so that the deflectable shaft portion or a distal tip of the catheter can be accurately positioned within the patient. In some embodiments, the actual image in the portion 901 can be compared with the predicted rendering in the portion 903 before delivering a treatment to the tissue. A fourth portion 904 can display the angular deflection of the adjusting knob 710 relative to the neutral orientation of the adjusting knob 710.

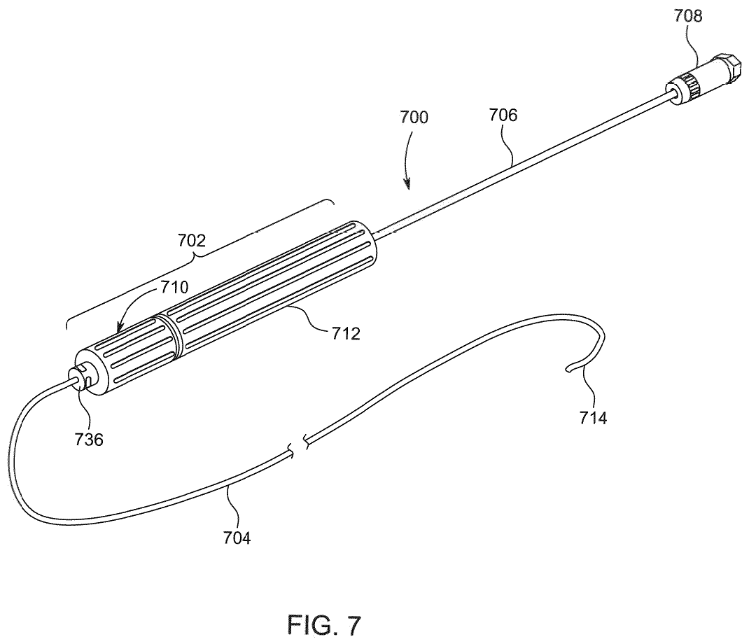

Referring FIG. 7 is an isometric view of a catheter 700. The catheter 700 includes a control handle 702 for a flexible tubular body 704. Throughout this specification, the term catheter is meant to include, without limitation, catheters, sheaths, and similar medical devices. As shown in FIG. 7, in one embodiment, the distal end of the handle 702 is connected to the catheter body 704 and the proximal end of the handle 702 is connected to tubing 706 that contains electrical wire and extends to an electrical connector 708. The handle 702 includes an adjusting knob 710 and a handle grip 712. As will become clear from this specification, the handle 702 is advantageous in that it is compact and allows a user to manipulate the catheter body's extreme distal end 714 in a bi-directional manner by pivoting the adjusting knob 710 relative to the handle grip 712 in one direction or the other about the longitudinal axis of the handle 702. Furthermore, in one embodiment, the handle 702 has a lumen that runs uninterrupted from the proximal end of the handle 702 to the extreme distal end 714 of the catheter body 704. This lumen can be used to provide contrast injection for guide wire insertion.

For a more detailed discussion of the handle 702, reference is now made to FIG. 8. FIG. 8 is an isometric exploded view of the handle 702. As shown in FIG. 8, the adjusting knob 710 is pivotally attached to a slide base 716 contained within the handle grip 712. To pivotally attach the adjusting knob 710 to the slide base 716, a dowel pin 718 is inserted into a pinhole 720 in the distal end of the slide base 716 and mates with a groove 722 in a hub portion 723 of the adjusting knob 710. A silicone O-ring 724 is disposed and retained between the hub portion 723 of the adjusting knob 710 and the distal end of the slide base 716.

As indicated in FIG. 8, a wire guide 726 is positioned within the adjusting knob 710 and is held in place by a retaining ring 728. A right slide member 730 and a left slide member 732 are slideably positioned within a slot (i.e., a slide compartment) 734 in the slide base 716. A catheter body-retaining nut 736 is used to secure the catheter body 704 to the distal end of the wire guide 726.

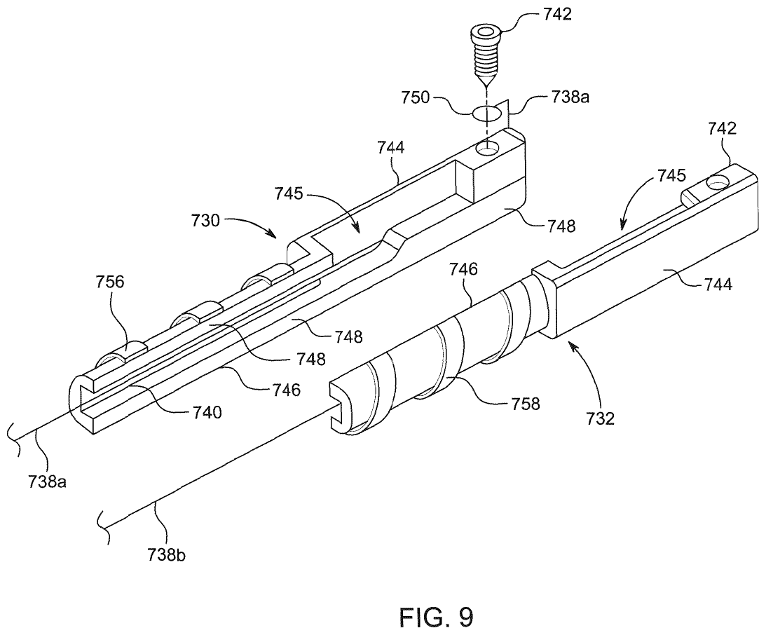

As illustrated in FIG. 9, a pair of deflection wires 738a, 738b extend distally from the right and left slide members 730, 732, respectively. Each of the deflection wires 738a, 738b, is affixed to the respective slide member 730, 732 via a respective retention screw 742. The slide members 730, 732 are mirror images of each other. Each of the slide members 730, 742 have a rectangular box-like proximal portion 744 and a half-cylinder distal portion 746. Each proximal portion 744 has a generally planar outer sidewall and bottom wall. These planar surfaces slideably displace against the generally planar sides and bottom of the slot 734, which act as thrust surfaces for the slide members 730, 732.

Each half-cylinder distal portion 746 is hollowed out along its longitudinal axis to form the passage 740 through which the deflection wires 738a, 738b extend when the slide members 730, 732 are in the assembled handle 702. Each of the slide members 730, 732 has a planar slide face 748 that slideably abuts against the planar slide face 748 of the opposing slide member 730, 732. As illustrated in FIG. 8, when the planar slide faces 748 of the slide members 730, 732 abut against each other and the extreme proximal ends of the slide members 730, 732 are flush with each other, the half-cylinder distal portions 746 of each of the slide members 730, 732 combine to form a complete cylinder with a channel or passage 740 there through.

As shown in FIG. 9, in one embodiment, the proximal end of each of the deflection wires 738a, 738b forms a loop 750 through which a retention screw 742 passes to secure the deflection wire 738a, 738b to the proximal portion of the respective slide member 730, 732. In one embodiment where the wires 738a, 738b only transmit tension forces, the wires 738a, 738b may deflect or flex within an open area 745 defined in the proximal portion 744 of each of the slide members 730, 732 when not under tension.

As shown in FIG. 9, in one embodiment, the outer circumference of the half-cylinder distal portion 746 of the right slide 730 is threaded with a right-hand thread 756, and the outer circumference of the half-cylinder distal portion 746 of the left slide 732 is threaded with a left-hand thread 758. In one embodiment, the outer circumference of the half-cylinder distal portion 746 of the right slide 730 is threaded with a left-hand thread, and the outer circumference of the half-cylinder distal portion 746 of the left slide 732 is threaded with a right-hand thread. Internal threads of the adjusting knob 710 mate with the corresponding external threads 756, 758 of the slide members 730, 732. More specifically, right internal threads of the adjusting knob 710 mate with the right external threads 756 of the right slide member 730 and the left internal threads of the adjusting knob 710 mate with the left external threads 758 of the left slide member 732. As the adjusting knob 710 is rotated clockwise relative to the longitudinal axis of the handle 702, the internal and external right threads engage, thereby causing simultaneous opposed displacement of the right and left slide members 730, 732 longitudinally within the slot 734 in the control handle 702. Specifically, because of the threading arrangement of the adjusting knob 710 and the slide members 730, 732, the right slide member 730 moves distally within the slot 734 and the left slide member 732 moves proximally within the slot 734 when the adjusting knob 710 is rotated clockwise relative to the handle grip 712 of the handle 702. Conversely, when the adjusting knob 710 is rotated in a counterclockwise manner relative to the handle grip 712 of the handle 702, the right slide member 730 moves proximally within the slot 734 and the left slide member 732 moves distally within the slot 734.

When the adjusting knob 710 is rotated such that the right slide member 730 is urged distally and the left slide member 732 is urged proximally, the deflection wire 738a connected to the right slide member 730 is placed into compression and the deflection wire 738b connected to the left slide member 732 is placed into tension. This causes the extreme distal end 714 of the catheter body 704 to deflect in a first direction. Conversely, when the adjusting knob 710 is rotated such that the right slide member 730 is urged proximally and the left slide member 732 is urged distally, the deflection wire 738a connected to the right slide member 730 is placed into tension and the deflection wire 738b connected to the left slide member 732 is placed into compression. This causes the extreme distal end 714 of the catheter body 704 to deflect in a second direction that is opposite the first direction.

The control handle 702 has several advantages. First, the control handle 702 is compact and may be operated with a single hand. Second, the threaded slide members 730, 732 and the adjusting knob 710 allow a physician to make fine, controlled adjustments to the bend in the distal end 714 of the catheter body 704. Third, once the adjusting knob 710 is rotated so as to cause a bend in the distal end 714 of the catheter body 704, the bend is maintained without requiring any action on the physician's part. Fourth, because the slide members 730, 732 simply displace distally and proximally along the longitudinal axis of the handle 702, they are less likely to permanently deform the wires 738a, 738b as compared to the wire displacement mechanisms in some prior art handles. Fifth, the control handle 702 is mechanically advantageous in that it provides increased deflection wire travel and reduced actuation effort for the physician, as compared to some prior art handles.

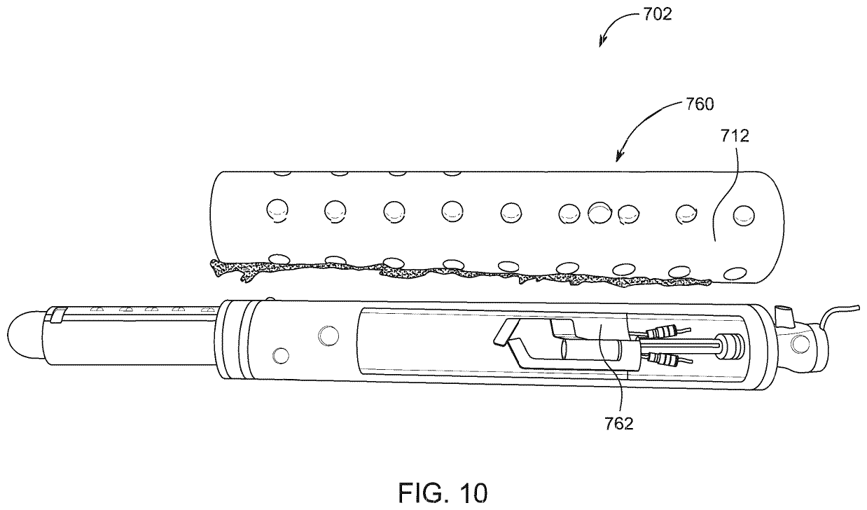

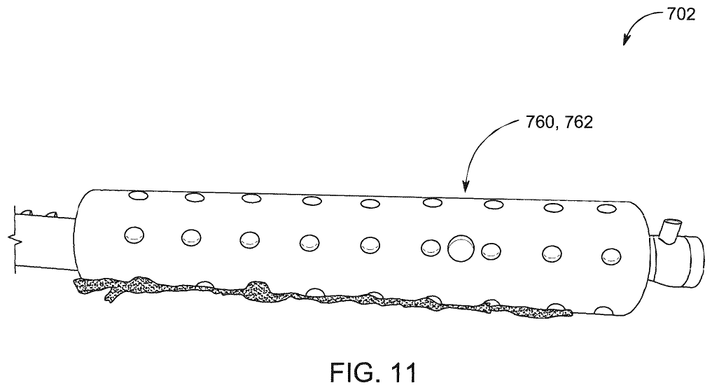

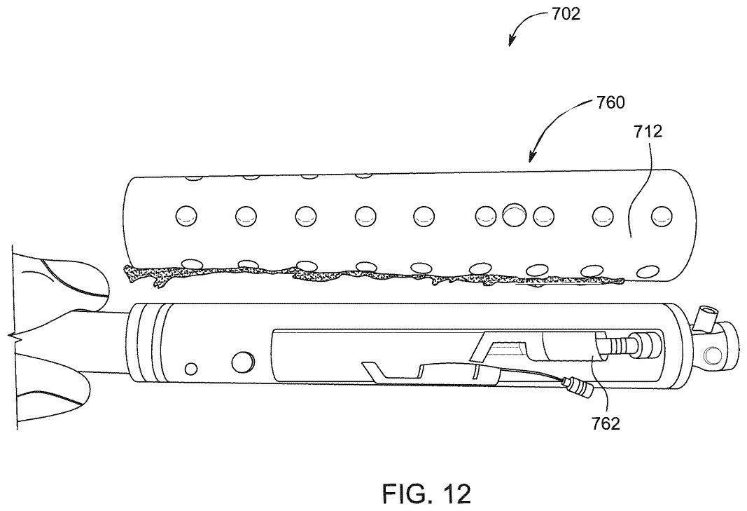

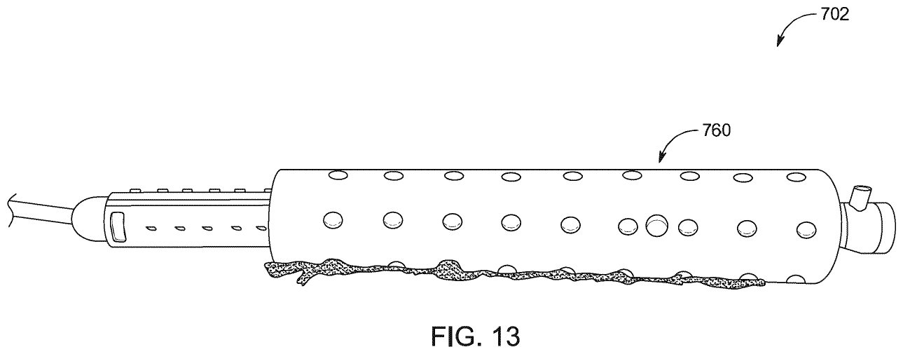

In some embodiments, the control handle 702 includes a visual position indicator configured to indicate when the slide members 730, 732 in one or more reference positions relative to the handle grip 712. For example, when aligned, the slide members 730, 732 are positioned to hold the distal ends of the deflection wires 738a, 738b relative to the control handle 702 to accommodate an undeflected configuration of the catheter body 704 suitable distal advancement or proximal retraction of the catheter body 704 when active steering of the distal end 714 via the control handle 702 is not desired. In some embodiments described herein, one of the slide members 730, 732 includes the visual position indicator and the handle grip 721 has an indication port that is aligned with the visual position indicator when the slide member 730, 732 are aligned in the neutral position. For example, FIG. 10 shows a photograph of a partially disassembled configuration of a prototype of an embodiment of the control handle 702 with an indication port 760 in the handle grip 712 and a visual position indicator 762 attached to the right slide member 730. In FIG. 10, the slide members 730. 732 are aligned in the neutral position and the handle grip 712 is shown offset to one side and the axial location of the indication port 760 is aligned with the visual position indicator 762. FIG. 11 is a photograph of the control handle of FIG. 10 illustrating visibility of the visual position indicator 762 through the indication port 760 when the slides are aligned in the neutral position. FIG. 12 is a photograph of a partially disassembled configuration of the control handle of FIG. 12 illustrating the relative positions of the indication port 760 and the visual position indicator 762 with the slides displaced from the neutral positions. FIG. 13 is a photograph of the control handle 702 of FIG. 13 illustrating non-visibility of the visual position indicator 762 through the indication port 760 when the slide members 730, 732 are displaced from the neutral positions. Any suitable alternate approach can also be used to incorporate a visual position indicator into the control handle 702.

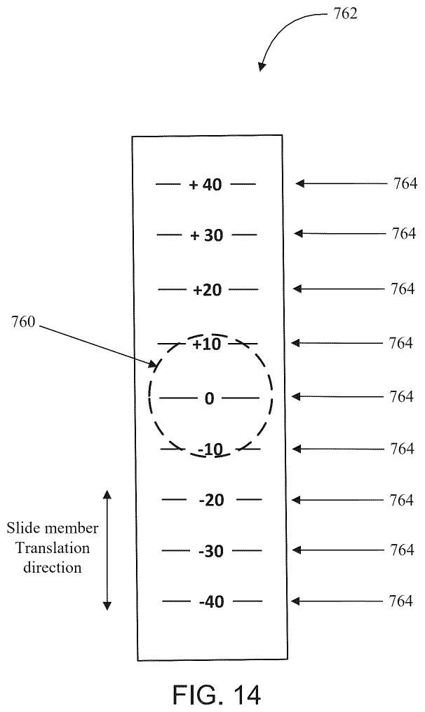

In some embodiments of the deflectable catheter assembly, the visual position indicator 762 includes deflection magnitude indications 764 as illustrated in FIG. 14. Each of the deflection magnitude indications 764 is visible through the indication port 760 when the slide members 730, 732 are positioned to induce an amount of deflection of the deflectable shaft section 714 corresponding to the deflection magnitude indication 764.

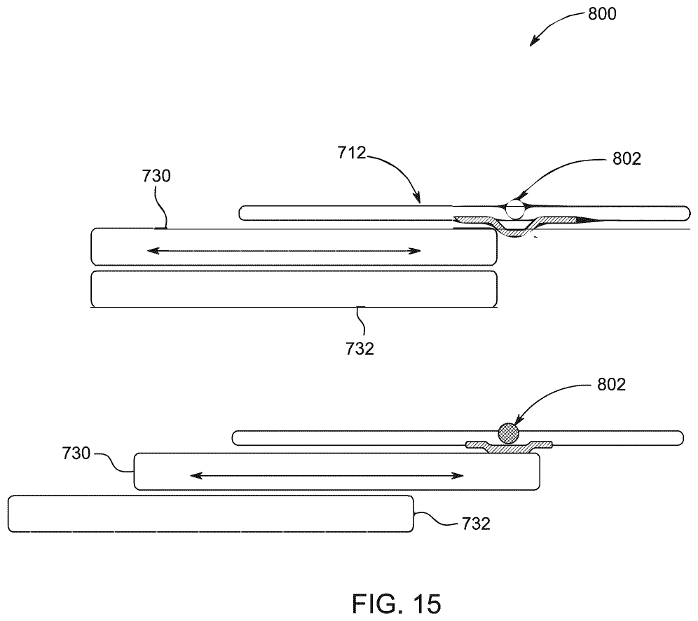

FIG. 15 illustrates a deflection feedback assembly 800, in accordance with embodiments. The deflection feedback assembly 800 includes a neutral position indicator light 802 and a switch 804. In the illustrated embodiment, the switch 804 is configured to be operated by the slide member 730 to supply power to the indicator light 802 when the slide members 730, 732 are not in their neutral positions. When the slide members 730, 732 are in their neutral positions, the switch 804 is open and the indicator light is off. Accordingly, the neutral position indicator light 802 is configured to provide feedback indicative of whether or not the slide members 730, 732 are in their neutral positions. In alternate embodiments, the switch 804 can be configured so that the indicator light 802 is on when the slide members 730, 732 are in their neutral positions and off when the slide members 730, 732 are not in their neutral positions.

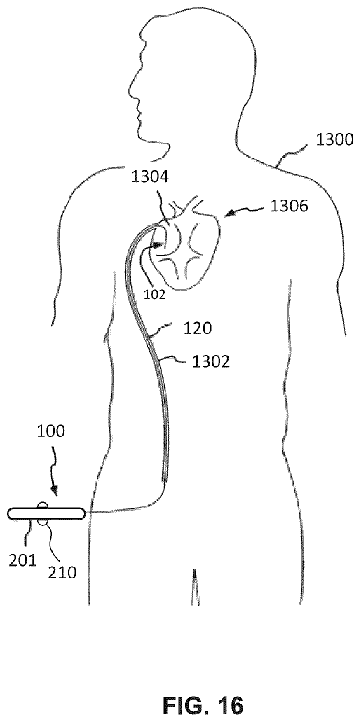

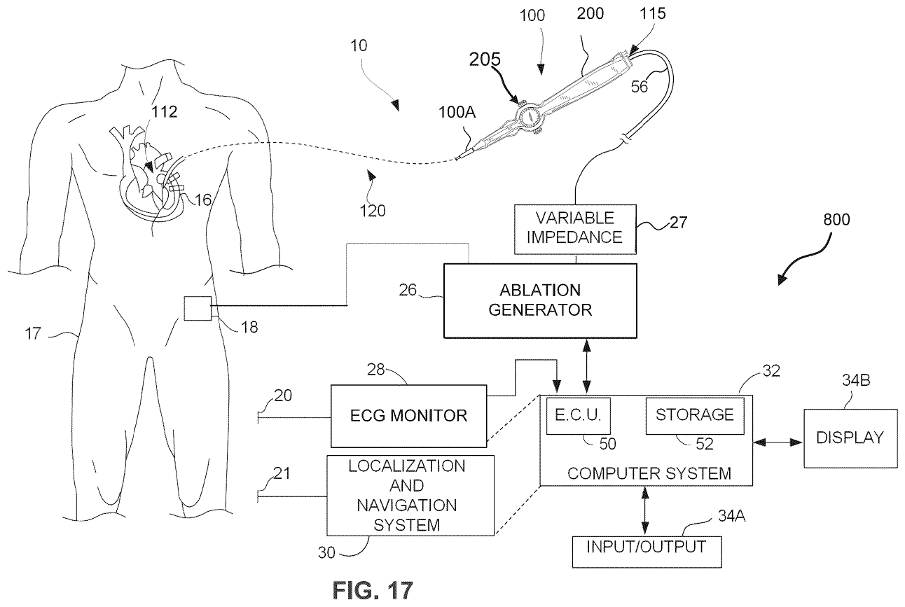

FIG. 16 is a diagrammatic illustration of the catheter 100 employed in a procedure on a patient 1300. Alternately, either of the catheters 200, 700 can be employed in the procedure on the patient 1300 the distal end 102 of the catheter 100 is inserted into the patient 1300 (e.g., intravenously via a body lumen 1302 of the patient 1300, percutaneously, or via other avenues for entering the patient's body). The distal end 102 of the catheter 100 is advanced until positioned in a selected location within the patient 1300 (e.g., within a chamber 1304 of the patient's heart 1306 or other organ, within a body cavity of the patient, etc.). The distal end of the catheter 100 is then deflected by rotating an outer actuator 210 about a transverse axis of the handle housing 201. As discussed herein, the handle housing 201 includes a deflection feedback assembly (e.g., 220 in FIG. 2) that converts the rotation of the outer actuator into a deflection signal indicative of a degree of deflection of the distal end 102 of the catheter 100 while the catheter shaft is within the patient. A system (e.g., as shown in FIG. 17) can be configured to predict a degree of deflection (e.g., an amount, directions, shape, etc.) and display such information to an operator. In some embodiments, this can include receiving the deflection signal, comparing the deflection signal to the deflection mapping table, and determining a deflection of the distal end 102 of the catheter 100. The degree of deflection, which can be based on the determined deflection of the distal end 102 of the catheter 100 can be displayed. The predicted degree of deflection can more accurately guide the operation on how to navigate the distal end 102 to appropriate location for treatment or diagnostic of parts (e.g., the heart 1306) of the patient 1306.

The catheter 100 can be any suitable catheter, such as a diagnostic catheter, an ablation catheter (e.g., an RF ablation catheter, an electroporation catheter), etc. Example electroporation systems and catheters for electroporation systems are discussed in PCT publication no. WO 2018/102376 A1, the entire disclosure of which is incorporated herein by reference.

FIG. 17 is a schematic and block diagram view of a system 10 that can be used for ablation (e.g., RF ablation or pulsed field ablation) to destroy abnormal cardiac tissue within the heart anatomy. In particular, system 10 includes an ablation catheter comprising deflection feedback, as discussed herein, to provide feedback related to an amount of deflection before, during or after an ablation treatment, for example.

The system 10 includes a catheter (e.g., 100, 700) having a distal shaft section (e.g., 145) coupled to a handle (e.g., 200, 400, 702). A connector 115 or an integrated cable (not illustrated) without a connector may be coupled at a proximal end of the handle 200 to provide mechanical and electrical connection(s) for cable(s) 56 extending from an ablation generator 26. The connector 115 may comprise conventional components known in the art and as shown is disposed at the proximal end of the catheter 100. The catheter 100 may also include other conventional components not illustrated herein such as a temperature sensor, additional electrodes, and corresponding conductors or leads disposable via the elongate shaft 120. In some embodiments, the diagnostic and/or therapeutic catheter can be configured for use in any suitable medical procedure such as, for example, cardiac mapping and/or ablation (e.g., PFA).

In some embodiments, the catheter 100 includes a diagnostic and/or therapeutic shaft assembly (i.e., FSA 140) attached to the distal shaft portion 145 of the deflectable shaft section 130. The diagnostic and/or therapeutic shaft assembly can have any suitable configuration for performing a diagnostic and/or therapeutic medical procedure. For example, in some embodiments, the diagnostic and/or therapeutic shaft assembly (i.e., FSA 140) includes the distal shaft section 145 with the electrodes 112 configured to accomplish a diagnostic and/or therapeutic medical procedure (e.g., the electrode shaft section 145). For example, the diagnostic and/or therapeutic shaft assembly comprising the electrode shaft section (e.g., 145) can include electrodes 112 that are electrically coupled to the generator 26 via suitable electrical wire or other suitable electrical conductors extending through the elongate shaft 120.

The electrodes 112 may be used for a variety of diagnostic and therapeutic purposes including, for example and without limitation, cardiac mapping and/or ablation (e.g., PFA). For example, and in some embodiments, the distal shaft portion 145 with the electrodes 112 may be configured as a bipolar electrode assembly for use in bipolar-based electroporation therapy. Specifically, electrodes 112 are individually electrically coupled to the generator 26 (e.g., via suitable electrical wire or other suitable electrical conductors extending through catheter shaft 120) and are configured to be selectively energized (e.g., by the generator 26 and/or computer system 32) with opposite polarities to generate a potential and corresponding electric field therebetween, for PFA therapy. That is, one of electrodes 112 is configured to function as a cathode, and the other is configured to function as an anode. Electrodes 112 may be any suitable electroporation electrodes. In the exemplary embodiment, electrodes 112 are ring electrodes. Electrodes 112 may have any other shape or configuration. It is realized that the shape, size, and/or configuration of electrodes 112 may impact various parameters of the applied electroporation therapy. For example, increasing the surface area of one or both electrodes 112 may reduce the applied voltage needed to cause the same level of tissue destruction. Further, the electrodes 112 on the distal shaft section 145 may be configured as a bipolar electrode assembly. In some embodiments, electrode assembly 100 may configured as a monopolar electrode assembly and use a patch electrode (e.g., return electrode 18) as a return or indifferent electrode.

The handle 200 is configured to be held by an operator (e.g., a clinician) and operable to articulate the deflectable section 130 of the catheter shaft 120. The handle 200 includes the outer actuator 210 (e.g., see FIG. 2) and a pull wire actuation mechanism that is drivingly coupled with the deflectable shaft section 130 via two pull wires (also referred as deflection wires) affixed onto a pull ring disposed at the distal portion of the deflectable shaft section 130. The pull wire actuation mechanism includes an input element that is articulable by the operator to articulate the pull wires to selectively curve the deflectable shaft section 130. The handle 200 can also include deflection feedback control (e.g., 220 in FIG. 2) configured to output a deflection signal indicative of a degree of deflection, as discussed herein.

A plurality of return electrodes designated 18, 20, and 21, which are diagrammatic of the body connections that may be used by the various sub-systems included in the overall system 10, such as an electroporation generator 26, an electrophysiology (EP) monitor such as an ECG monitor 28, a localization and navigation system 30 for visualization, mapping and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode. The system 800 may further include a main computer system 32 (including an electronic control unit 50 and data storage-memory 52), which may be integrated with system 30 provided for visualization, mapping, and navigation of internal body structures in certain embodiments. The computer system 32 may further include conventional interface components, such as various user input/output mechanisms 34a and a display 34b, among other components.

In many embodiments, the electronic control unit 50 of the computer system 32 can be a controller. The controller 50 can be coupled via the one or more cables (e.g., 56) of the connector 115 to the handle and the display 34b. The controller 50 is configured to determine, based on deflection mapping data and the output signals, the deflection amounts of the deflectable shaft section, the deflection mapping data being a predetermined relationship between the output signals and the deflection amounts. The controller 50 is configured to send an input probing current to the first terminal (e.g., 303 in FIG. 3A and FIG. 3C) of the variable resistor (e.g., 300 in FIG. 3A and FIG. 3C). The controller 50 can be further configured to receive, via the second terminal (e.g., 304 in FIG. 3A and FIG. 3C) of the variable resistor, an output current after the input probing current passes through the resistance element (e.g., 301 in FIG. 1). Based on the output signal and the deflection mapping data, the controller can determine the degree of deflection of the deflectable shaft section.

The generator 26 may be configured to energize the electrode element(s) in accordance with a RF ablation or an electroporation energization strategy, which may be predetermined or may be user-selectable. For electroporation, a variable impedance 27 allows the impedance of the system to be varied to limit arcing from the catheter electrode of catheter (e.g., 100). Moreover, variable impedance 27 may be used to change one or more characteristics, such as amplitude, duration, pulse shape, and the like, of an output of the generator 26. Although illustrated as a separate component, variable impedance 27 may be incorporated in the catheter 100 or generator 26. In some embodiments, each variable impedance 27 may be connected to a different catheter electrode or group of catheter electrodes to allow the impedance through each catheter electrode or group of catheter electrodes to be separately varied. Additional details of an example electroporation systems are discussed in PCT publication no. WO 2018/102376 A1, the entire disclosure of which is incorporated herein by reference.

In one or more embodiments of the present disclosure, A deflectable catheter assembly including an elongate catheter shaft includes a deflectable shaft section; and a handle coupled to the elongate catheter shaft, and one or more pull wires. The handle includes a housing; a deflection control coupled to the housing and operable to deflect the deflectable shaft section; and a deflection feedback assembly disposed within the housing. The deflection feedback assembly is coupled to the deflection control and configured to convert motion of the deflection control into a signal indicative of a degree of deflection of the deflectable shaft section. The one or more pull wires are coupled to the deflection control and extending through the elongate catheter shaft. The one or more pull wires are operable to induce deflection of the deflectable shaft section. Optionally, the deflection feedback assembly includes a variable resistor configured to vary resistance values upon operation of the deflection control. The variable resistor is coupled to the deflection control such that a first position of the deflection control corresponds to a first resistance value of the variable resistor and a second position of the deflection control corresponds to a second resistance value of the variable resistor. Optionally, the first resistance value corresponds to a first degree of deflection of the deflectable shaft section; and the second resistance value corresponds to a second degree of deflection of the deflectable shaft section. Optionally, the variable resistor includes: a resistance element; a movable contact configured to move along the resistance element in response to operation of the deflection control to cause a change in a resistance value of the resistance element; a first terminal coupled to the resistance element to pass in an input electrical current through the resistance element; and a second terminal coupled to the movable contact, the second terminal configured to output an output electrical current indicative of the degree of deflection based on the resistance value corresponding to a position of the movable contact along the resistance element. Optionally, the variable resistor is a rotary variable resistor. The resistance element is an arc-shape resistance element. The movable contact is radially movable along the resistance element in response to operation of the deflection control to cause a change in a resistance value of the resistance element. Optionally, the variable resistor is a linear variable resistor. The resistance element is a linear resistance element. The movable contact is slidable along the resistance element in response to operation of the deflection control to cause a change in a resistance value of the resistance element. Optionally, the deflection feedback assembly includes an electrical wire extending proximally away from the elongate catheter shaft. Optionally, the degree of deflection is characterized by a deflection amount and a deflection direction. Optionally, the degree of deflection of the deflectable shaft section is determined based on the signal from the deflection feedback assembly. Optionally, the handle further includes an inner platform disposed within the housing of the handle. The inner platform includes a first side and a second side opposite to the first side. The deflection control and the one or more pull wires are coupled to the first side of the inner platform. The deflection feedback assembly is disposed on the second side of the inner platform. Optionally, the deflection feedback assembly has a form factor corresponding to a shape of the second side of the inner platform and a thickness configured to fit within a space between the second side of the inner platform and an interior surface of the housing of the handle. Optionally, the handle further includes a chassis slidable within the housing of the handle. The one or more pull wires are coupled to the chassis to cause deflection of the deflectable shaft section as the chassis is moved. The deflection feedback assembly is coupled to the chassis such that the movement of the chassis is converted to the signal indicative of the degree of deflection. Optionally, the deflection feedback assembly includes a variable resistor having a circular shape. Optionally, the deflection feedback assembly includes at least one of a magneto-resistor configured to cause change in a resistance upon rotating the deflection control. Optionally, the deflection feedback assembly includes a printed circuit board includes a contact coupled to the deflection control. The printed circuit board configured to receive, via the contact, a position of the deflection control and convert the position to the degree of deflection of the deflectable shaft section.

In one or more embodiments of the present disclosure, a bidirectional deflectable catheter assembly includes: an elongate catheter shaft includes a deflectable shaft section; a handle coupled to the elongate catheter shaft; and one or more pull wires. The handle includes: a housing; a rotatable knob coupled exterior to the housing and operable to deflect the deflectable shaft section; and a variable resistor configured to vary resistance values upon rotation of the rotatable knob. Each resistance value corresponds to a degree of deflection of the deflectable shaft section. The one or more pull wires are coupled to the rotatable knob and extending through the elongate catheter shaft and operable to induce deflection of the deflectable shaft section in towards a first side or a second side with respect to a home position. Optionally, the handle further includes an inner platform disposed within the housing of the handle; the inner platform includes a first side and a second side opposite to the first side; the rotatable knob and the one or more pull wires are coupled to the first side of the inner platform; and the variable resistor is disposed on the second side of the inner platform. Optionally, the variable resistor has a form factor corresponding to a shape of the second side of the inner platform and a thickness configured to fit within a space between the second side of the inner platform and an interior surface of the housing of the handle. Optionally, the variable resistor is a rotary variable resistor includes: an arc-shape resistance element; a movable contact radially movable along the arc-shape resistance element in response to rotating of the rotatable knob to cause a change in a resistance value along the arc-shape resistance element; a first terminal coupled to the arc-shape resistance element to pass an input electrical current through the arc-shape resistance element; and a second terminal coupled to the movable contact, the second terminal configured to output an output electrical current indicative of the degree of deflection based on the resistance value corresponding to a position of the movable contact along the resistance element. Optionally, the variable resistor includes an electrical wire extending proximally away from the elongate catheter shaft. Optionally, the variable resistor has a circular shape. Optionally, the variable resistor is disposed exterior to the housing and coupled to the rotatable knob.

In one or more embodiments of the present disclosure, a catheter system includes: an elongate catheter shaft includes a deflectable shaft section; a handle coupled the elongate catheter shaft; and one or more pull wires. The handle includes: a housing having a distal end and a proximal end, the distal end being configured to receive the elongate catheter shaft; a deflection control coupled to the housing and operable to deflect the deflectable shaft section; and a deflection feedback assembly disposed within the housing and coupled to the deflection control, the deflection feedback assembly configured to provide output signals indicative of deflection amounts of the deflectable shaft section in response to operation of the deflection control. The one or more pull wires coupled to the deflection control and extending through the elongate catheter shaft and operable to induce deflection of the deflectable shaft section; a connector coupled at the proximal end of the housing and configured to pass one or more cables; and a controller coupled via the one or more cables of the connector to the handle and a display. The controller is configured to determine, based on deflection mapping data and the output signals, the deflection amounts of the deflectable shaft section. The deflection mapping data is a predetermined relationship between the output signals and the deflection amounts. Optionally, the output signals are characterized by a variable parameter; and the deflection mapping data includes a first parameter value corresponds to a first deflection amount of the deflectable shaft section; and a second parameter value corresponds to a second deflection amount of the deflectable shaft section. Optionally, the variable parameter is at least one of: an electrical resistance, an electrical current, a voltage, or a magnetic strength. Optionally, the deflection feedback assembly is a variable resistor or a variable inductor. Optionally, the variable resistor includes: a resistance element; a movable contact configured to move along the resistance element in response to operation of the deflection control to cause a change in a resistance value of the resistance element; a first terminal coupled to the resistance element to pass in an input electrical current through the resistance element; and a second terminal coupled to the movable contact. The second terminal is configured to output an output electrical current indicative of the degree of deflection based on the resistance value corresponding to a position of the movable contact along the resistance element. Optionally, the controller is configured to: send an input probing current to the first terminal of the variable resistor; receive, via the second terminal of the variable resistor, an output current after the input probing current passes through the resistance element; and determine the degree of deflection of the deflectable shaft section based on the output signal and the deflection mapping data. Optionally, the variable resistor is a rotary variable resistor.

In one or more embodiments of the present disclosure, a method of determining a shape of a deflectable shaft section of an elongate catheter shaft coupled to a handle is described. The method includes: receiving, via the handle, a first degree of deflection of the deflectable shaft section of the elongate catheter shaft in a body of a patient. The handle includes: a housing; a deflection control operable to deflect the deflectable shaft section, the deflection control in a first position, the first position corresponding to the first degree of deflection of the deflectable shaft section; and a deflection feedback assembly disposed within the housing and configured to convert a position of the deflection control into a signal indicative of a degree of deflection of the deflectable shaft section. The method further includes receiving, via the deflection feedback assembly, a first signal indicating that the deflection control is in the first position. The method further includes receiving a second position of the deflection control. The second position corresponds to a second deflection of the deflectable shaft section. The method further includes receiving, via the deflection feedback assembly, a second signal indicating that the deflection control having the second deflection; and generating, via a processor, a visual representation of the deflectable shaft section, the visual representation indicating the deflectable shaft section having the second degree of deflection. Optionally, the method further includes determining, using deflection mapping data, the first degree of deflection and the second degree of deflection based on the first signal associated with the first position and the second signal associated with the second position of the deflection control, the deflection mapping data indicating a relationship between the signals and the deflection amounts. Optionally, the method further includes generating the deflection mapping data based on signals of the deflection feedback assembly, positions of the deflection control, and degrees of deflection of the deflectable shaft section. Optionally, generating the deflection mapping data includes: measuring, via the deflection feedback assembly, a signal at each of a number of predetermined positions of the deflection control on the handle; measuring a deflection amount of the deflectable shaft section at each of the predetermined positions; and generating a deflection mapping table relating each of the signals and each associated deflection amounts and linking each signal to each associated predetermined position.

In one or more embodiments of the present disclosure, a deflectable catheter assembly includes: an elongate catheter shaft includes a deflectable shaft section; one or more pull wires, wherein each of the one or more pull wires extends through the elongate catheter shaft and is actuatable to induce deflection of the deflectable shaft section; and a control handle coupled to the elongate catheter shaft. The control handle includes: one or more slide members coupled with the one or more pull wires, wherein at least one of the one or more slide members includes a visual position indicator configured to indicate one or more reference positions of the one or more slide members; a rotatable deflection control knob drivingly coupled with the one or more slide members and rotatable to induce translation the one or more slide members to actuate the one or more pull wires; and a housing in which the one or more slide members are slidably disposed. The housing includes an indication port through which the visual position indicator is visible when the one or more slide members are in at least one of the one or more reference positions of the one or more slide members. Optionally, the visual position indicator is visible through the indication port when the one or more slide members are in a neutral position in which the one or more pull wires do not induce deflection of the deflectable shaft section in the absence of any external contact forces being applied to the deflectable shaft section. Optionally, the visual position indicator is not visible through the indication port when the one or more slide members are in a translated position in which the one or more pull wires induce deflection of the deflectable shaft section. Optionally, the visual position indicator includes deflection magnitude indications; and each of the deflection magnitude indications is visible through the indication port when the one or more slide members are positioned to induce an amount of deflection of the deflectable shaft section corresponding to the deflection magnitude indication. Optionally, the one or more slide members include two slide members. Optionally, the one or more pull wires include two pull wires; each of the two pull wires is coupled to a respective one of the two slide members; and one of the two slide members includes the visual position indicator.

It is to be understood that terms such as “distal,” “proximal,” “top,” “bottom,” “front,” “side,” “length,” “inner,” and the like that can be used herein merely describe points of reference and do not necessarily limit embodiments of the present disclosure to any particular orientation or configuration. As used herein, “proximal” refers to a direction toward the end of the catheter near an operator (e.g., a clinician) and “distal” refers to a direction away from the operator and (generally) inside the body of a patient. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

The terms “longitudinal,” “axial” or “axially” are generally longitudinal as used herein to describe the relative position related to a catheter, a catheter handle, or other components of the system herein. For example, “longitudinal” or “axial” indicates an axis passing along a center of a catheter from a proximal end to a distal end, or along a center of the catheter handle from a proximal end to a distal end. The term “radial” generally refers to a direction perpendicular to the “axial” direction.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

While certain embodiments have been described, these embodiments have been presented by way of example only and are not intended to limit the scope of the present disclosures. Indeed, the novel methods, apparatuses and systems described herein can be embodied in a variety of other forms; furthermore, various omissions, substitutions, and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A deflectable catheter assembly comprising:
an elongate catheter shaft comprising a deflectable shaft section;
a handle coupled to the elongate catheter shaft, the handle comprising:
a housing;
a deflection control coupled to the housing and operable to deflect the deflectable shaft section; and
a deflection feedback assembly disposed within the housing, the deflection feedback assembly coupled to the deflection control and configured to convert motion of the deflection control into a signal indicative of a degree of deflection of the deflectable shaft section; and
one or more pull wires coupled to the deflection control and extending through the elongate catheter shaft, the one or more pull wires operable to induce deflection of the deflectable shaft section.

2. The deflectable catheter assembly of claim 1, wherein:
the deflection feedback assembly comprises a variable resistor configured to vary resistance values upon operation of the deflection control; and
the variable resistor is coupled to the deflection control such that a first position of the deflection control corresponds to a first resistance value of the variable resistor and a second position of the deflection control corresponds to a second resistance value of the variable resistor.

3. The deflectable catheter assembly of claim 2, wherein:
the first resistance value corresponds to a first degree of deflection of the deflectable shaft section; and
the second resistance value corresponds to a second degree of deflection of the deflectable shaft section.

4. The deflectable catheter assembly of claim 2, wherein the variable resistor comprises:
a resistance element;
a movable contact configured to move along the resistance element in response to operation of the deflection control to cause a change in a resistance value of the resistance element;
a first terminal coupled to the resistance element to pass in an input electrical current through the resistance element; and
a second terminal coupled to the movable contact, the second terminal configured to output an output electrical current indicative of the degree of deflection based on the resistance value corresponding to a position of the movable contact along the resistance element.

5. The deflectable catheter assembly of claim 4, wherein:
the variable resistor is a rotary variable resistor;
the resistance element is an arc-shape resistance element; and
the movable contact is radially movable along the resistance element in response to operation of the deflection control to cause a change in a resistance value of the resistance element.

6. The deflectable catheter assembly of claim 4, wherein:
the variable resistor is a linear variable resistor;
the resistance element is a linear resistance element; and
the movable contact is slidable along the resistance element in response to operation of the deflection control to cause a change in a resistance value of the resistance element.

7. The deflectable catheter assembly of claim 1, wherein the deflection feedback assembly comprises an electrical wire extending proximally away from the elongate catheter shaft.

8. The deflectable catheter assembly of claim 1, wherein the degree of deflection is characterized by a deflection amount and a deflection direction.

9. The deflectable catheter assembly of claim 1, wherein the degree of deflection of the deflectable shaft section is determined based on the signal from the deflection feedback assembly.

10. The deflectable catheter assembly of claim 1, wherein:
the handle further comprises an inner platform disposed within the housing of the handle;
the inner platform comprises a first side and a second side opposite to the first side;
the deflection control and the one or more pull wires are coupled to the first side of the inner platform; and
the deflection feedback assembly is disposed on the second side of the inner platform.

11. The deflectable catheter assembly of claim 10, wherein the deflection feedback assembly has a form factor corresponding to a shape of the second side of the inner platform and a thickness configured to fit within a space between the second side of the inner platform and an interior surface of the housing of the handle.

12. The deflectable catheter assembly of claim 1, wherein:
the handle further comprises a chassis slidable within the housing of the handle;
the one or more pull wires are coupled to the chassis to cause deflection of the deflectable shaft section as the chassis is moved; and
the deflection feedback assembly is coupled to the chassis such that a movement of the chassis is converted to the signal indicative of the degree of deflection.

13. The deflectable catheter assembly of claim 1, wherein the deflection feedback assembly comprises a variable resistor having a circular shape.

14. The deflectable catheter assembly of claim 1, wherein the deflection feedback assembly comprises at least one of a magneto-resistor configured to cause change in a resistance upon rotating the deflection control.

15. The deflectable catheter assembly of claim 1, wherein:
the deflection feedback assembly comprises a printed circuit board comprising a contact coupled to the deflection control; and
the printed circuit board configured to receive, via the contact, a position of the deflection control and convert the position to the degree of deflection of the deflectable shaft section.

16. A bidirectional deflectable catheter assembly comprising:
an elongate catheter shaft comprising a deflectable shaft section;
a handle coupled to the elongate catheter shaft, the handle comprising:
a housing;
a rotatable knob coupled exterior to the housing and operable to deflect the deflectable shaft section; and
a variable resistor configured to vary resistance values upon rotation of the rotatable knob, wherein each resistance value corresponds to a degree of deflection of the deflectable shaft section; and
one or more pull wires coupled to the rotatable knob and extending through the elongate catheter shaft and operable to induce deflection of the deflectable shaft section in towards a first side or a second side with respect to a home position.

17.-22. (canceled)

23. A catheter system comprising:
an elongate catheter shaft comprising a deflectable shaft section;
a handle coupled the elongate catheter shaft, the handle comprising:
a housing having a distal end and a proximal end, the distal end being configured to receive the elongate catheter shaft;
a deflection control coupled to the housing and operable to deflect the deflectable shaft section; and
a deflection feedback assembly disposed within the housing and coupled to the deflection control, the deflection feedback assembly configured to provide output signals indicative of deflection amounts of the deflectable shaft section in response to operation of the deflection control; and
one or more pull wires coupled to the deflection control and extending through the elongate catheter shaft and operable to induce deflection of the deflectable shaft section;
a connector coupled at the proximal end of the housing and configured to pass one or more cables; and
a controller coupled via the one or more cables of the connector to the handle and a display, the controller configured to determine, based on deflection mapping data and the output signals, the deflection amounts of the deflectable shaft section, the deflection mapping data being a predetermined relationship between the output signals and the deflection amounts.

24. The catheter system of claim 23, wherein:
the output signals are characterized by a variable parameter; and
the deflection mapping data comprises a first parameter value corresponds to a first deflection amount of the deflectable shaft section; and a second parameter value corresponds to a second deflection amount of the deflectable shaft section.

25. The catheter system of claim 24, wherein the variable parameter is at least one of: an electrical resistance, an electrical current, a voltage, or a magnetic strength.

26. The catheter system of claim 23, wherein the deflection feedback assembly is a variable resistor or a variable inductor.

27.-39. (canceled)
